(19) United States
(12) Patent Application Publication
(10) Pub. No.: US 2023/0285394 A1
(43) Pub. Date: Sep. 14, 2023

(54) METHODS OF TREATMENT OF NON-SMALL-CELL LUNG CARCINOMA USING TELISOTUZUMMAB VEDOTIN AND OSIMERTINIB

(71) Applicant: AbbVie Biotherapeutics Inc., North Chicago, IL (US)

(72) Inventors: Vincent Blot, Seattle, WA (US); Janet Yikai Jin, Foster City, CA (US); Philip B. Komarnitsky, Chestnut Hill, MA (US)

(21) Appl. No.: 18/182,165

(22) Filed: Mar. 10, 2023

Related U.S. Application Data
(60) Provisional application No. 63/318,987, filed on Mar. 11, 2022.

(51) Int. Cl. A61K 31/506; A61K 47/68; C07K 16/28; A61P 35/04

(52) U.S. Cl. CPC A61K 31/506; A61K 47/6803; C07K 16/2863; A61P 35/04; A61K 2039/505

(57) ABSTRACT

The present disclosure provides improved methods of treatment of NSCLC cancers using the combination of telisotuzumab vedotin and osimertinib.

### 1. SEQUENCE LISTING

The instant application contains a Sequence Listing which has been submitted electronically in .xml format and is hereby incorporated by reference in its entirety. Said .xml copy, created on Mar. 9, 2023, is named 350794-41001-Sequence-Listing.xml and is 26,510 bytes in size.

### 2. TECHNICAL FIELD

The present application pertains to, among other things, improved methods of treatment of non-small-cell lung carcinoma using the combination of telisotuzumab vedotin (Teliso-V; ABBV-399) and osimertinib, and methods of selecting specific patient populations for treatment.

### 3. BACKGROUND

c-Met is a signaling tyrosine kinase receptor expressed on the surface of epithelial and endothelial cells. Activation of c-Met by hepatocyte growth factor (HGF), its only known ligand, has been shown to control cell proliferation, angiogenesis, survival, and cellular motility (Ma et al., 2003, Cancer Metastasis Rev., 22:309-325; Gherardi et al., 2012, Nat Rev Cancer., 12:89-103). Deregulation of c-Met signaling via receptor upregulation has been implicated in the development of non-small-cell lung cancer (NSCLC) (Ma et al., 2005, Cancer Res., 65:1479-1488; Spigel et al., 2013, J Clin Oncol., 31:4105-4114; The Cancer Genome Atlas Research Network, 2014, Nature, 511:543-550).

NSCLC represents 85% of all lung cancers and is the leading cause of cancer-related death worldwide (GLOBOCAN, 2018; American Cancer Society: Cancer Facts and FIGS. 2018). Aberrant c-Met signaling is common in NSCLC and is believed to occur via multiple mechanisms. Deregulated c-Met signaling has been associated with poor prognosis (Cappuzzo et al., 2009, J Clin Oncol., 27(10):1667-1674; Vuong et al., 2018, Lung Cancer., 123:76-82; Tong et al., 2016, Clin Cancer Res., 22(12):3048-3056), tumorigenesis, resistance to chemotherapy/radiotherapy (Gu et al., 2016, J Hematol Oncol., 9:66-68) and acquired resistance to epidermal growth factor receptor (EGFR) tyrosine kinase inhibitors (TKI) (Turke et al., 2010, Cancer Cell., 17(1):77-88).

The first-in-class ADC telisotuzumab vedotin (Teliso-V), was created by linking the anti-c-Met humanized monoclonal antibody ABT-700 to monomethyl auristatin E (MMAE) via a valine-citrulline linker (ABT-700-vcMMAE). ABT-700 has been shown to specifically target Teliso-V to c-Met-expressing tumor cells with high affinity (Wang et al., 2016, BMC Cancer., 16:105-119; Wang et al., 2017, Clin Cancer Res., 23:992-1000). Teliso-V has demonstrated promising antitumor activity in preclinical studies in cells overexpressing c-Met, independent of MET amplification status, potentially expanding the target population for this drug to patients whose tumors express c-Met (Wang et al., 2017, Clin Cancer Res., 23:992-1000).

Osimertinib is becoming the standard treatment for metastatic NSCLC with EGFR sensitizing mutation with or without T790M mutation. Despite initial response, tumors become resistant to osimertinib due to emergence of clones that acquire secondary resistance mechanisms such as MET amplification, which was shown to be acquired in among approximately 20% patients upon development of resistance to osimertinib (Ramalingam et al., 2018, Annals of Oncology, 29 (Suppl 8):2018).

### 4. SUMMARY

Provided herein are methods for treating c-Met overexpressing non-squamous NSCLC tumors in a subject that exhibits an EGFR mutation, wherein the subject was previously treated with osimertinib, and the NSCLC had progressed while the subject was on osimertinib.

Further provided herein are methods of treating non-squamous NSCLC tumors that overexpress c-Met in a human subject, wherein the subject received previous osimertinib therapy and experienced progressive disease while on the osimertinib therapy, comprising determining whether the tumor exhibits c-Met overexpression, and if the tumor tissue exhibits c-Met overexpression, administering the combination of osimertinib and Teliso-V, wherein the non-squamous NSCLC carries a mutated EGFR gene.

Also provided are methods of treating non-squamous NSCLC tumors that express c-Met in a plurality of human subjects, comprising the steps of determining whether the tumor exhibits: i) c-Met overexpression or ii) lack of c-Met overexpression; if the tumor tissue exhibits lack of c-Met overexpression, excluding the subjects having the tumor that exhibits lack of c-Met overexpression from treatment; if the tumor tissue exhibits c-Met overexpression, selecting the subjects for treatment and administering to the selected subjects the combination of osimertinib and Teliso-V, wherein the non-squamous NSCLC tumor carries a mutated EGFR gene.

Also provided herein are methods for treating a subject having non-squamous NSCLC with an EGFR mutation comprising administering a therapeutically effective amount of a combination of Teliso-V and osimertinib to the subject if the NSCLC overexpresses c-Met.

### 5. BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-ID show representative cytoplasmic staining intensities for c-Met on non-squamous NSCLC. SP44 OptiView IHC Cytoplasmic Staining Intensities (20X) of IHC score of 0 (FIG. 1A); 1+(FIG. 1B); 2+(FIG. 1C); and 3+(FIG. 1D).

FIGS. 2A-2D show representative membranous staining intensities for c-Met on non-squamous NSCLC. SP44 OptiView IHC Membranous Staining Intensities 20X of IHC score of 0 (FIG. 2A); 1+(FIG. 2B); 2+(FIG. 2C); and 3+(FIG. 2D).

FIGS. 3A1-3C3, FIGS. 3B1-3B3, and FIGS. 3C1-3C3 show representative membranous staining intensities for c-Met on non-squamous NSCLC. SP44 OptiView IHC Membranous Staining Intensities 20X of c-Met Negative (FIGS. 3A1-3A3); c-Met Positive (FIGS. 3B1-3B3); and c-Met High (FIGS. 3C1-3C3).

FIG. 4 shows distribution of SP44 UltraView and OptiView IHC at 3+ intensity staining on a commercial cohort of NSCLC.

FIG. 5 shows that ≥25% 3+ cutoff for SP44 OptiView, selects similar patient population as SP44 Ultraview. The clinical modeling data was obtained from the Teliso-V monotherapy trial NCT02099058. Abbreviations shown in FIG. 5 and determination of the values in the table are as follow: PPA (positive percent agreement): #oV positive uV positive/Total uV positive; NPA (Negative percent agreement): #oV negative uV negative/Total uV negative; OPA (Overall percent agreement): #oV positive uV positive+#oV negative uV negative/Total number of samples tested; PPV (Positive predictive value): #of oV positive uV positive/total #of oV positive; NPV (Negative predictive value): #of oV negative uV negative/Total oV negative. % BOR represents the percent best overall response.

FIG. 6 shows that >50% 3+ cutoff for SP44 OptiView, selects similar patient population as SP44 Ultraview. The clinical modeling data was obtained from the Teliso-V monotherapy trial NCT02099058. Abbreviations shown in FIG. 6 and determination of the values in the table are as follow: PPA (positive percent agreement): #oV positive uV positive/Total uV positive; NPA (Negative percent agreement): #oV negative uV negative/Total uV negative; OPA (Overall percent agreement): #oV positive uV positive+#oV negative uV negative/Total number of samples tested; PPV (Positive predictive value): #of oV positive uV positive/total #of oV positive; NPV (Negative predictive value): #of oV negative uV negative/Total oV negative. % BOR represents the percent best overall response.

### 6. DETAILED DESCRIPTION

### 6.1. Anti-c-Met ADC: Telisotuzumab Vedotin

As described throughout the specification, telisotuzumab vedotin (Teliso-V) is an ADC comprised of the c-Met targeting antibody ABT-700 (PR-1266688, h224G11) conjugated to the potent cytotoxin monomethyl auristatin E (MMAE) through a valine citrulline (vc) linker.

Conjugation to ABT-700 is via a thioether linkage formed with a sulfhydryl group of a cysteine residue of ABT-700. The production and biological activities of ABT-700 are described in U.S. Pat. No. 8,741,290. The production and biological activities of telisotuzumab vedotin are described in U.S. Pat. No. 10,603,389.

Telisotuzumab vedotin, as used herein, refers to an ADC having the following structural formula:

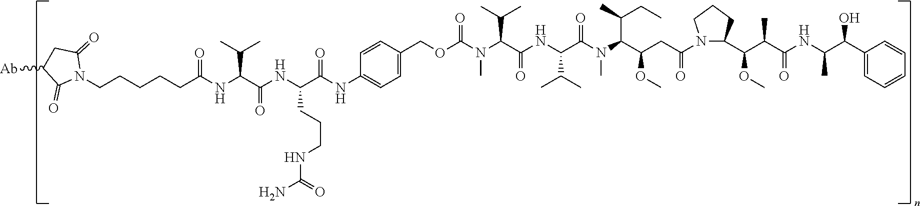

wherein n is 2 or 4, the Ab is ABT-700, and conjugation of the drug to the antibody is via a linkage formed with a sulfhydryl group of a cysteine residue of ABT-700. In a preferred embodiment, n has a value of 2. In a preferred embodiment, n has a value of 4. The purification and characterization of telisotuzumab vedotin with n equal to 2 or 4 is described in U.S. Pat. No. 10,603,389.

Telisotuzumab vedotin has been used in a Phase 1 clinical trial (see Example 16 of U.S. Pat. No. 10,603,389) in a pharmaceutical formulation with a DAR of about 2.4 to 3.6, for example, 3.1.

Telisotuzumab vedotin can be used at a 1:1 E2/E4 ratio, which corresponds to an average DAR of 3.0 or about 3.0. In other words, telisotuzumab vedotin is used as a composition comprising a 1:1 ratio of the E2 and E4 purified fractions of antibody-drug conjugate. In other alternative embodiments, telisotuzumab vedotin can be used at a DAR of 2.9.

ABT-700, as used herein, refers to any antibody having the following heavy and light chain sequences. The heavy chain of ABT-700 comprises (constant regions are bold; CDRs are underlined (Kabat-numbered CDR sequences disclosed as SEQ ID NOS: 1-3, respectively, in order of appearance)):

| | |
|---|---|
| QVOLVQSGAE VKKPGASVKV SCKASGYIFT AYTMHWVRQA PGQGLEWMGW | 050 |
| IKPNNGLANY AQKFQGRVTM TRDTSISTAY MELSRLRSDD TAVYYCARSE | 100 |
| ITTEFDYWGQ GTLVTVSSAS TKGPSVFPLA PSSKSTSGGT AALGCLVKDY | 150 |
| FPEPVTVSWN SGALTSGVHT FPAVLQSSGL YSLSSVVTVP SSSLGTQTYI | 200 |
| CNVNHKPSNT KVDKRVEPKS CDCHCPPCPA PELLGGPSVF LFPPKPKDTL | 250 |
| MISRTPEVTC VVVDVSHEDP EVKFNWYVDG VEVHNAKTKP REEQYNSTYR | 300 |
| VVSVLTVLHQ DWLNGKEYKC KVSNKALPAP IEKTISKAKG QPREPQVYTL | 350 |
| PPSREEMTKN QVSLTCLVKG FYPSDIAVEW ESNGQPENNY KTTPPVLDSD | 400 |
| GSFFLYSKLT VDKSRWQQGN VFSCSVMHEA LHNHYTQKSL SLSPG | 445 |

(variable region sequence disclosed as SEQ ID NO:4) (full-length sequence disclosed as SEQ ID NO:5)
and the light chain of ABT-700 comprises (CDR sequences disclosed as SEQ ID NOS:6-8, respectively, in order of appearance):

| | |
|---|---|
| DIVMTQSPDS LAVSLGERAT INCKSSESVD SYANSFLHWY QQKPGQPPKL | 050 |
| LIYRASTRES GVPDRFSGSG SGTDFTLTIS SLQAEDVAVY YCQQSKEDPL | 100 |
| TFGGGTKVEI KRTVAAPSVF IFPPSDEQLK SGTASVVCLL NNFYPREAKV | 150 |
| QWKVDNALQS GNSQESVTEQ DSKDSTYSLS STLTLSKADY EKHKVYACEV | 200 |
| THQGLSSPVT KSFNRGEC | 218 |

(variable region sequence disclosed as SEQ ID NO:9) (full-length sequence disclosed as SEQ ID NO: 10)

In one embodiment, the ABT-700 heavy chain is encoded by the following nucleotide sequence (full-length sequence disclosed as SEQ ID NO: 11):

ATGGGATGGTCTTGGATCTTTCTGCTGTTTCTGTCTGGTACTGCTGGTGTG
CTGAGCcaggtccagctggtgcaatccggcgcagaggtgaagaagccaggc
gcttccgtgaaggtgagctgtaaggcctctggctacatcttcacagcatac
accatgcactgggtgaggcaagctcctgggcagggactggagtggatggga
tggattaaacccaacaatgggctggccaactacgcccagaaattccagggt
agggtcactatgacaagggataccagcatcagcaccgcatatatggagctg
agcaggctgaggtctgacgacactgctgtctattattgcgccaggagcgaa
attacaacagaattcgattactgggggcagggcaccctggtgaccgtgtcc
tctgccagcaccaagggcccaagcgtgttccccctggcccccagcagcaag
agcaccagcggcggcacagccgccctgggctgcctggtgaaggactacttc
cccgagcccgtgaccgtgtcctggaacagcggagccctcacttctggagtt
cataccttcccagcagtattgcagagcagtggcctgtattcactgtcttcc
gtcgtaacagttccatcctccagcctcgggacacagacttacatttgtaac
gtgaatcacaagcctagcaacaccaaggtcgacaagagagttgaaccaaag
agttgtgattgccactgtcctccctgcccagctcctgagctgcttggcggt
cccagtgtcttcttgtttccccctaaacccaaagacaccctgatgatctca
aggactcccgaggtgacatgcgtggtggtggatgtgtctcatgaggaccca
gaggtgaagttcaactggtacgtggacggcgtggaggtgcacaacgccaag
accaagcccagagaggagcagtacaacagcacctacagggtggtgtccgtg
ctgaccgtgctgcaccaggactggctgaacggcaaggagtacaagtgtaag
gtgtccaacaaggccctgccagccccaatcgaaaagaccatcagcaaggcc
aagggccagccaagagagccccaggtgtacaccctgccacccagcagggag
gagatgaccaagaaccaggtgtccctgacctgtctggtgaagggcttctac
ccaagcgacatcgccgtggagtgggagagcaacggccagcccgagaacaac
tacaagaccacccccccagtgctggacagcgacggcagcttcttcctgtac
agcaagctgaccgtggacaagagcagatggcagcagggcaacgtgttcagc
tgctccgtgatgcacgaggccctgcacaaccactacacccagaagagcctg
agcctgtccccaggctga

Secretion signal peptide in bold CAPITAL letters; includes final stop codon (TGA); constant region is bold; CDRs are underlined (CDR sequences disclosed as SEQ ID NOS: 12-14, respectively, in order of appearance)

In one embodiment, the ABT-700 light chain is encoded by the following nucleotide sequence (full-length sequence disclosed as SEQ ID NO: 15):

ATGGAAACTGATACACTGCTGCTGTGGGTCCTGCTGCTGTGGGTCCCTGGA
AGCACAGGGgacattgtgatgacccagtctcccgatagcctggccgtgtcc
ctgggcgagagggctaccatcaactgtaaaagctccgaatctgtggactct
tacgcaaacagctttctgcactggtatcagcaaaagccaggccaacctcca
aagctgctgatttacagggcttctaccagggagagcggcgtgcccgatagg
ttcagcggatctggcagcggcaccgactttacactgaccatctccagcctg
caggccgaagatgtggcagtctattactgccagcagtccaaggaggacccc
ctgactttcgggggtggtactaaagtggagatcaagcgtacggtggccgct
cccagcgtgttcatcttccccccaagcgacgagcagctgaagagcggcacc
gccagcgtggtgtgtctgctgaacaacttctaccccagggaggccaaggtg
cagtggaaggtggacaacgccctgcagagcggcaacagccaggagagcgtc
accgagcaggacagcaaggactccacctacagcctgagcagcaccctgacc
ctgagcaaggccgactacgagaagcacaaggtgtacgcctgtgaggtgacc
caccagggcctgtccagccccgtgaccaagagcttcaacaggggcgagtgc
tga

Secretion signal peptide in bold CAPITAL letters; includes final stop codon (tga); constant region is bold; CDRs are underlined (CDR sequences disclosed as SEQ ID NOS: 16-18, respectively, in order of appearance).

### 6.2. Telisotuzumab Vedotin Compositions

Teliso-V is provided as an aqueous composition suitable for administration via intravenous infusion. In some embodiments, the aqueous composition comprises 20 mg/mL Teliso-V, 10 mM histidine buffer, pH 6.0, 7% (w/v) sucrose, 0.03% (w/v) polysorbate 80. The composition may be in the form of a lyophilized powder that, upon reconstitution with 5.2 mL sterile water or other solution suitable for injection or infusion (for example, 0.9% saline, Ringer's solution, lactated Ringer's solution, etc.) provides the above aqueous composition.

### 6.3. Osimertinib

Osimertinib is a kinase inhibitor for oral use that binds irreversibly to certain mutant forms of EGFR (T790M, L858R, and exon 19 deletions). The molecular formula for osimertinib mesylate is C₂₈H₃₃N₇O₂·CH4O3S, and the molecular weight is 596 g/mol. The chemical name is N-(2-{2-dimethylaminoethyl-methylamino}-4-methoxy-5-{[4-(1-methylindol-3-yl)pyrimidin-2-yl]amino} phenyl)prop-2-enamide mesylate salt. Osimertinib has the following structural formula:

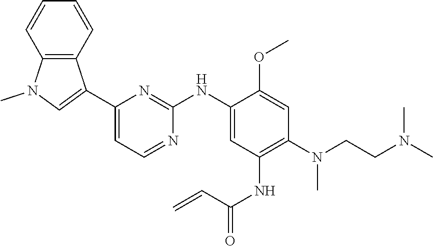

Osimiternib, sold under the brand name Tagrisso®, is currently indicated for adjuvant therapy after tumor resection for non-small cell lung cancer (NSCLC) in patients whose tumors have epidermal growth factor receptor (EGFR) exon 19 deletions or exon 21 L858R mutations, first-line treatment for patients with metastatic NSCLC whose tumors have EGFR exon 19 deletions or exon 21 L858R mutations, and for metastatic EGFR T790M mutation-positive NSCLC in patients whose disease has progressed on or after EGFR tyrosine kinase inhibitor (TKI) therapy. Osimertinib is provided in tablet form for oral administration in tablets containing 40 or 80 mg of osimertinib.

### 6.4. Teliso-V and Osimertinib Combination Methods of Use

Methods described herein involve treating patients with the combination of Teliso-V and osimertinib who have metastatic/locally advanced non-squamous NSCLC with documented EGFR mutations del19 or L858R, with or without T790M mutation and none of the EFGR mutations known to be resistant to osimertinib and in which c-Met is overexpressed. In embodiments, Teliso-V is administered once every two weeks (14 days) at 1.6 mg/kg or 1.9 mg/kg. Teliso-V may be given before or after osimertinib oral dosing. Patients receive osimertinib daily at a dose of 40 mg or 80 mg per day, administered orally. In an embodiment, 1.9 mg/kg Teliso-V is administered once every two weeks, and 80 mg of osimertinib is administered daily. In an embodiment, 1.6 mg/kg Teliso-V is administered once every two weeks, and 80 mg of osimertinib is administered daily. In embodiments, administration of the combination of Teliso-V and osimertinib is continued until either disease progression or unacceptable toxicity occurs. In embodiments, administration of the combination of Teliso-V and osimertinib is continued for 4 months, 5 months, 6 months, 7 months, 8 months, 12 months, 18 months, 24 months, or longer.

Efficacy of the combination of Teliso-V and osimertinib is assessed with respect to standard of care therapy, where statistically significant superior results of the combination compared to the standard of care are indicative of efficacy. In embodiments, the standard of care is a chemotherapy based regimen. In embodiments, subjects treated with the combination of Teliso-V and osimertinib have an objective response rate (ORR) greater than or equal to 25%. In embodiments, efficacy of the treatment includes a median duration of response (DoR) of 4 months or more (e.g, of at least 4 months, at least 6 months, at least 8 months, and/or at least 10 months). Other efficacy endpoints include progression free survival (PFS), overall survival (OS), and an acceptable safety and tolerability profile.

Results from the Stage 1 Interim 4 analysis of an ongoing Phase 2 study (NCT03539536) of Teliso-V monotherapy at a dose of 1.9 mg/kg in c-Met positive (≥25% 3+ IHC staining) advanced non-squamous NSCLC subjects previously treated with chemotherapy (or ineligible for chemotherapy) and at least one prior EGFR tyrosine kinase inhibitor treatment demonstrated an ORR (defined as either complete response or partial response) in non-squamous NSCLC EGFR mutant patients of 11.6%. In the subset of those non-squamous NSCLC EGFR mutant subjects who were known to have been previously treated with osimertinib and experienced progressive disease while on osimertinib, the ORR was 0%. In contrast, based on the January 2022 interim results from the phase 1 NCT02099058 trial, treatment of non-squamous NSCLC EGFR mutant subjects who had been previously treated with osimertinib and experienced progressive disease while on osimertinib with the combination of 1.9 mg/kg Teliso-V once every two weeks and 80 mg osimertinib daily resulted in an ORR of 66.7%. In the April 2022 interim results, treatment of non-squamous NSCLC EGFR mutant subjects who had been previously treated with osimertinib and experienced progressive disease while on osimertinib with the combination of 1.9 mg/kg Teliso-V once every two weeks and 80 mg osimertinib daily resulted in an ORR of 53%. The combination of 1.6 mg/kg Teliso-V once every two weeks and 80 mg osimertinib daily resulted in an ORR of 42.9%. Thus, the administration of two therapeutics, neither of which is effective on its own in the subset of the NSCLC EGFR mutant patient population that experienced progressive disease while on prior osimertinib therapy, provides an effective treatment in that patient population.

In a preferred embodiment, treatment of non-squamous NSCLC subjects with c-Met overexpression and EGFR mutant status who progressed while on previous osimertinib therapy achieves an ORR, progression free survival (PFS) and/or overall survival (OS) that is superior to that of the standard of care. In embodiments, the standard of care is a chemotherapy-based regimen. For example, in embodiments, the combination of Teliso-V and osimertinib achieves an ORR greater than 25%, greater than 30%, greater than 35%, greater than 40%, greater than 45%, greater than 50%, greater than 55%, greater than 60%, greater than 65%, or greater than 70% or more. In other embodiments, treatment with the combination of Teliso-V and osimertinib results in PFS of at least 4 months or more, or an OS of 13, 14, 15, 16, 17, or 19 months or more.

Treatment with the combination of Teliso-V and osimertinib of non-squamous NSCLC subjects with c-Met overexpression and EGFR mutant status who progressed while on previous osimertinib therapy will demonstrate a favorable safety profile with less than 20%, optionally less than 15% or less than 10%, of adverse events, i.e., peripheral neuropathy, neutropenia, pneumonitis and ocular toxicity, leading to discontinuation of treatment with the combination of Teliso-V and osimertinib. Subjects that experience an adverse event of 3+ or greater, will be less than 20%, optionally less than 15%, or less than 10%.

IHC assays are known to one of ordinary skill in the art for assessing the expression level of a target protein (see Diagnostic Immunohistochemistry: Theranostic and Genomic Applications by David Dabbs (5th edition, 2019), and Companion and Complementary Diagnostics: From Biomarker Discovery to Clinical Implementation (2019), editor JT Jorgensen). c-Met IHC assays are used in the methods of this disclosure to assess overexpression levels of c-Met in tumor tissue from a subject having non-squamous NSCLC. A preferred method for determining c-Met overexpression levels is the c-Met IHC assay described in detail in Example 1 and is referred to herein as the “c-Met Teliso-V staining protocol.”

In one embodiment, the method of treatment comprises the steps of determining the level of c-Met overexpression within the non-squamous NSCLC tumor by performing c-Met immunohistochemistry (IHC) on tumor tissue (i.e., obtained from a biopsy, resection or cytology sample; the tumor tissue can be archival tumor tissue or fresh tumor tissue) from the subject, and further comprising the step of determining whether the tumor tissue exhibits c-Met overexpression or a lack of c-Met overexpression.

Based on the presence or lack of c-Met overexpression in the tumor as determined by a c-Met IHC assay, the decision to treat the subject who progressed while on previous osimertinib therapy with a combination of Teliso-V and osimertinib is made. In one embodiment, subjects having tumors that are EGFR mutant and exhibit c-Met overexpression are treated with the combination of Teliso-V at a dose of 1.6 or 1.9 mg/kg intravenously once every two weeks and osimertinib at a dose of 80 mg taken orally daily. In another embodiment, subjects having tumors that are EGFR mutant and lacking c-Met overexpression are excluded from treatment with the combination of Teliso-V and osimertinib.

In one embodiment, a population of subjects who progressed while on previous osimertinib therapy having non-squamous NSCLC tumors with mutant EGFR status are treated or not treated according to c-Met overexpression levels of their tumor tissue as determined by c-Met IHC. In embodiments, the sub-population of subjects having a tumor that is EGFR mutant with c-Met overexpression is treated with combination of Teliso-V at a dose of 1.6 or 1.9 mg/kg intravenously once every two weeks and osimertinib at a dose of 80 mg taken orally daily, while the sub-population having a tumor without c-Met overexpression is excluded from treatment.

In another embodiment, an individual subject who progressed while on previous osimertinib therapy having a tumor that is EGFR mutant with c-Met overexpression is treated with combination of Teliso-V at a dose of 1.6 or 1.9 mg/kg intravenously once every two weeks and osimertinib at a dose of 80 mg taken orally daily, while an individual subject having a tumor without c-Met overexpression is excluded from treatment.

In embodiments, subjects treated with a combination of Teliso-V and osimertinib achieve one or more of the following clinical endpoints: overall response rate (ORR) greater than or equal to 25%; a median duration of response (DoR) of at least 4 months; progression free survival (PFS) of at least 5 months; or overall survival (OS) of at least 16 months, and exhibit stable disease (SD); partial response (PR); or confirmed response (CR) per RECIST version 1.1.

As will be appreciated by those of skill in the art, the recommended dosage for Teliso-V may need to be adjusted to optimize patient response and maximize therapeutic benefit. For example, the dosage of Teliso-V may be reduced to 1.6 mg/kg to manage peripheral neuropathy.

In one embodiment, the dosage for subjects weighing over 100 kg is calculated as if they weighed 100 kg. In this embodiment, the maximum dosage is 190 mg. Similarly, the recommended dosage for osimertinib may need to be adjusted to optimize patient response and maximize therapeutic benefit. For example, the dosage of osimertinib may be reduced to 40 mg daily.

### 6.5. Patient Selection

Patients treated with the combination of Teliso-V and osimertinib have c-Met-overexpressing NSCLC tumors of the non-squamous type. Patients are selected for treatment with Teliso-V based on their known EGFR status, prior treatment status, and c-Met overexpression level as determined by immunohistochemistry (c-Met IHC).

In an embodiment, some or all of the following inclusion and exclusion criteria of TABLES 1 and 2 are used to determine whether a subject is eligible for treatment:

TABLE 1
Key Inclusion Criteria

Age ≥ 18 years
c-Met overexpressing (OE) NSCLC assessed by a designated
immunohistochemistry (IHC) laboratory
Subjects with histologically documented metastatic/locally
advanced c-Met + NSCLC harboring EGFR mutations del19,
or L858R, with or without T790M mutation.
Locally advanced or metastatic NSCLC with measurable disease
per Response Evaluation Criteria In Solid Tumors (RECIST) v1.1
Received at least one but no more than 2 prior lines of systemic
therapy in the locally advanced or metastatic setting. One of
the prior lines of therapy must have contained osimertinib, and
the subject must have had disease progression while on osimertinib.
No more than one prior lime nay have contained chemotherapy.
Consecutive EGFR tyrosine kinase inhibitor treatments are
counted as 1 regimen.
Eastern Cooperative Oncology Group (ECOG) performance status
0-1 or 0-2 and adequate bone marrow, renal, and hepatic function.

TABLE 2
Key Exclusion Criteria

Adenosquamous histology
EGFR mutations known to be resistant to osimertinib
History of a major immunologic reaction to any IgG-containing agent
Major surgery within 21 days prior to the first dose of Teliso-V
Anticancer therapy within 21 days or herbal therapy/strong cytochrome
P450 3A4 inhibitors within 7 days prior to the first dose of Teliso-V
History of interstitial lung disease or pneumonitis requiring systemic
steroid treatment. Subject has evidence of pulmonary fibrosis on
screening imaging assessment or any history of pneumonitis or
interstitial lund disease IILD) within 3 months of the planned first
dose of the study drug.
Subject has uncontrolled metastases to the CNS based on head CT or
MRI. Subjects with brain metastases may be eligible at least 2 weeks
after definitive therapy to all known sites of CNS disease provided
they are asymptomatic and either off or on a non-increasing dose
(in last 2 weeks) of systemic steroids and not on anticonvulsants
for seizure activity directly related to progressive CNS metastases.
Subject has received radiation therapy to the lung <6 months prior
to the first dose of Teliso-V.
Subject has a clinically significant condition(s) including but not
limited to the following:
Grade ≥2 edema or lymphedema.
Grade ≥2 ascites or pleural effusion; asymptomatic pleural effusion
following surgical intervention such as indwelling pleural catheter
placement is allowed.
Grade ≥2 neuropathy.
Active uncontrolled bacterial or viral infection.
Symptomatic congestive heart failure.
Unstable angina pectoris or cardiac arrhythmia.
Extensive metastatic liver disease involving ≥50% of the liver in
the judgment of the Investigator or Medical Monitor or sum of longest
diameters of RECIST version 1.1 measurable liver lesions ≥10 cm.
Psychiatric illness/social situation that would limit compliance.
History of hypersensitivity to active or inactive excipients of osimertinib.
History of osimertinib dose reduction to below 80 mg QD
Refractory nausea and vomiting, chronic gastrointestinal diseases,
inability to swallow the formulated product, or previous significant
bowel resection that would preclude adequate absorption of osimertinib
Any of the following cardiac criteria: a) Mean resting corrected QT
interval (QTc) >470 ms; b) Any clinically important abnormalities in
rhythm, conduction, or morphology of resting ECG, e.g., complete left
bundle branch block, second-or third-degree heart block, PR interval
>250 ms; c) Any factors that increase the risk of QTc prolongation
or risk of arrhythmic events such as heart failure, hypokalemia,
congenital long QT syndrome, or any concomitant medication
known to prolong the QT interval.

### 6.5.1. Selection Criteria: NSCLC Tumor Type

Patients selected for treatment are patients with c-Met-overexpressing non-squamous NSCLC. Patients are selected for treatment based on their known EGFR status, prior treatment status, and c-Met expression level as determined by immunohistochemistry (IHC).

In some embodiments, patients selected for treatment with the combination of Teliso-V and osimertinib have metastatic/locally advanced non-squamous NSCLC with documented EGFR mutations del19 or L858R, with or without T790M mutation and none of the EFGR mutations known to be resistant to osimertinib. The decision on whether to select a particular patient for treatment with the combination of Teliso-V and osimertinib requires determining whether the patient's NSCLC has cells carrying a mutation of the Epidermal Growth Factor Receptor gene (EGFR).

In embodiments, patients selected for treatment have a c-Met-overexpressing non-squamous NSCLC that carries at least one EGFR mutation. In some embodiments, the at least one EGFR mutation is selected from an exon 19 deletion, an exon 21 L858R mutation, with or without a T790M mutation. In some embodiments, the at least one EGFR mutation is detected by an FDA-approved test.

One such test uses real-time polymerase chain reaction (PCR) to identify at least 42 mutations in exons 18, 19, 20 and 21 of the EGFR gene. The test has been clinically validated in multiple clinical trials as a companion diagnostic (CDx) for both first- and second-line EGFR TKI therapy in patients with advanced NSCLC (Heeke, et al., (2019) Clinical Lung Cancer, 21 (1): 56-65).

Next genome sequencing (NGS) can also be used to detect EGFR mutations. A number of companies have FDA approved CDx assays to detect EGFR mutations, including Foundation One CDx, Thermo Fisher Oncomine NSCLC and Guardant 360CDx. See also Ding et al., (2019) Thoracic Cancer 10: 1879-1884 discussing the use of the Thermo Fisher Oncomine NSCLC assay.

In certain embodiments, subjects are divided into groups based on whether or not there is c-Met overexpression as determined by IHC and EGRF status (wild type or mutated). Decisions to treat are made with respect to each of the resulting groups.

### 6.5.2. Selection Criteria: Prior Treatment

In some embodiments, subjects selected for treatment have received no more than 2 lines of prior systemic therapy (including no more than 1 line of systemic cytotoxic chemotherapy in the locally advanced or metastatic setting. Consecutive EGFR tyrosine kinase inhibitor treatments are counted as 1 regimen. In some embodiments, the cytotoxic chemotherapeutic is a platinum chemotherapeutic such as cisplatin, oxaliplatin, and carboplatin, or a platinum-based doublet, such as cisplatin/pemetrexed, carboplatin/pemetrexed, carboplatin/paclitaxel.

In some embodiments, the cytotoxic chemotherapeutic is a taxane based chemotherapeutic such as paclitaxel, albumin paclitaxel, or docetaxel.

In embodiments, subjects selected for treatment are those who have progressed on a prior regimen that comprised osimertinib therapy. Subjects that have progressed on a regiment comprising osimertinib therapy, may have been 1) treated with a regimen comprising chemotherapy prior to being treated with the regimen comprising osimertinib, 2) treated with a regimen comprising chemotherapy after being treated with the regimen comprising osimertinib, or 3) not treated with a regimen comprising chemotherapy. As an example, subjects receiving chemotherapy that are selected for treatment are those who have 1) progressed on a prior regimen comprising osimertinib therapy, and 2) received a prior regimen comprising platinum chemotherapy, including platinum doublet chemotherapy.

In embodiments, treatment of a subject without regard to whether or not the subject had received prior osimertinib therapy is contemplated, wherein the method comprises administering to a human subject having non-squamous NSCLC the combination of osimertinib and Teliso-V, wherein the NSCLC carries a mutated EGFR gene and overexpresses c-Met. In embodiments, the method comprises treating a subject without regard to whether or not they had received prior osimertinib therapy. In embodiments, the method comprises treating a subject who did not receive prior osimertinib therapy or did not progress while on prior osimertinib therapy. In an embodiment, the method comprises comprising administering to a human subject having non-squamous NSCLC the combination of osimertinib and Teliso-V, wherein the NSCLC carries a mutated EGFR gene and overexpresses c-Met, wherein the subject had not received prior osimertinib therapy, or wherein the subject had not progressed while on prior osimertinib therapy.

### 6.5.3. Selection Criteria: c-Met Expression by IHC

IHC is used to evaluate candidate patients for selection for treatment with the combination of Teliso-V and osimertinib on the basis of observed c-Met overexpression in non-squamous NSCLC tumor tissue. In some embodiments, c-Met IHC is performed on at least one non-squamous NSCLC tumor tissue from the subject, wherein the tumor tissue is selected from archival tumor tissue and/or fresh tumor tissue.

In one embodiment, c-Met overexpression or lack of c-Met overexpression within the non-squamous NSCLC tumor is determined by performing c-Met immunohistochemistry (c-Met IHC) on neoplastic cells from tumor tissue from the subject and determining the level of c-Met expression. In embodiments, c-Met overexpression is defined as a c-Met expression level of 1) ≥25% of neoplastic cells from tumor tissue assessed by c-Met IHC have ≥1+ membrane and/or cytoplasmic staining; 2) ≥25% of neoplastic cells from tumor tissue assessed by c-Met IHC have 2+ membrane and/or cytoplasmic staining; 3) ≥25% of neoplastic cells from tumor tissue assessed by c-Met IHC have 3+ membrane and/or cytoplasmic staining; or 4) ≥50% of neoplastic cells from tumor tissue assessed by c-Met IHC have 3+ membrane and/or cytoplasmic staining.

In embodiments, lack of c-Met overexpression is defined as a c-Met expression level of 1)<25% of neoplastic cells from tumor tissue assessed by c-Met IHC have ≥1+ membrane and/or cytoplasmic staining; 2)<25% of neoplastic cells from tumor tissue assessed by c-Met IHC have 2+ membrane and/or cytoplasmic staining; 3)<25% of neoplastic cells from tumor tissue assessed by c-Met IHC have 3+ membrane and/or cytoplasmic staining; or 4)<50% of neoplastic cells from tumor tissue assessed by c-Met IHC have 3+ membrane and/or cytoplasmic staining.

In some embodiments, the c-Met IHC is performed according to the c-Met Teliso-V Staining Protocol.

Various c-Met expression levels can be determined by c-Met immunohistochemistry. In embodiments, patients selected for treatment are those who have a c-Met expression level of 1) ≥25% of neoplastic cells from tumor tissue assessed by c-Met IHC have ≥1+ membrane and/or cytoplasmic staining; 2) ≥25% of neoplastic cells from tumor tissue assessed by c-Met IHC have 2+ membrane and/or cytoplasmic staining; 3) ≥25% of neoplastic cells from tumor tissue assessed by c-Met IHC have 3+ membrane and/or cytoplasmic staining; or 4) ≥50% of neoplastic cells from tumor tissue assessed by c-Met IHC have 3+ membrane and/or cytoplasmic staining

In certain embodiments, subjects having non-squamous NSCLC with EGFR mutation exhibiting a lack of c-Met overexpression are excluded from treatment with the combination of Teliso-V and osimertinib. In an embodiment, patients selected for exclusion from treatment are those who have a c-Met expression level of 1)<25% of neoplastic cells from tumor tissue assessed by c-Met IHC have ≥1+ membrane and/or cytoplasmic staining; 2)<25% of neoplastic cells from tumor tissue assessed by c-Met IHC have 2+ membrane and/or cytoplasmic staining; 3) <25% of neoplastic cells from tumor tissue assessed by c-Met IHC have 3+ membrane and/or cytoplasmic staining; or 4)<50% of neoplastic cells from tumor tissue assessed by c-Met IHC have 3+ membrane and/or cytoplasmic staining.

### 6.5.3.1. IHC Determination of c-Met Expression

c-Met specific immunohistochemistry (c-Met IHC) is contemplated as a means for determining c-Met-overexpression in the NSCLC of a candidate patient for treatment with Teliso-V. For this purpose, IHC scores of 0, 1+, 2+, and 3+ describe the visual c-Met staining intensities for individual neoplastic cells from tumor tissue, according to:
0=no staining
1+=weak staining
2+=moderate staining
3+=strong staining.
   Typically, there are ˜100 human NSCLC cells in a 20x fixed field. IHC scoring as used herein refers to the intensity of membranous c-Met staining or the intensity of membrane and/or cytoplasmic c-Met staining.

In some embodiments, patients having NSCLC selected for treatment with Teliso-V have c-Met-overexpression determined by IHC, wherein said IHC comprises the steps of:
1) staining the membrane and/or cytoplasm of neoplastic cells of the NSCLC with a diagnostic reagent specific for c-Met, and
2) scoring the intensity of membrane or membrane and/or staining, wherein IHC scoring is performed using values scaled to correspond to a score of 0, a score of 1+, a score of 2+, and/or a score of 3+,
   wherein said score of 0 corresponds to a minimal visual membrane and/or cytoplasm staining intensity of at or about the intensity of a negative control, said score of 3+ corresponds to a maximum visual membrane and/or cytoplasm staining intensity of at or about the intensity of a positive control, and said scores of 1+ and 2+ correspond to visual membrane and/or cytoplasm staining intensities of at or about ⅓, and of at or about ⅔, of the maximum visual membrane and/or cytoplasm staining intensity of the positive control, respectively.

Details on how to visualize and determine the level of c-Met overexpression are presented below and in Example 1. For purposes of this application, including the claims, the particular assay used in Example 1 is referred to as the “c-Met Teliso-V staining protocol.” Briefly, a c-Met IHC staining assay for c-Met overexpression was developed using the Ventana c-Met CONFIRM (SP44) kit (Catalog Number 790-4430) which uses the UltraView detection system, and is contemplated for use in the selection of patients for treatment with Teliso-V. In this assay, tissue samples are stained with the Ventana anti-c-Met antibody and then scored by determining the percentages of neoplastic cells of the tumor tissue that stain at certain intensity levels from weak/low to strong/high (i.e., 0, 1+, 2+, to 3+). This assay produces staining of the c-Met protein both in the cytoplasm and in the cell membrane, of which the membranous staining and/or cytoplasmic staining is used in IHC score determination.

If different c-Met IHC scoring results are obtained with different IHC methods, then the c-Met IHC scoring results determined with the methods described in Example 1 are those to be used in determining whether a particular embodiment falls within the scope of the embodiments. For example, for evaluating expression of the c-Met protein one would use the “c-Met Teliso-V staining protocol.” If the reagents used in this protocol are no longer available, another FDA-approved protocol for assessment of c-Met expression levels by IHC can be used.

IHC Detection of c-Met

c-Met IHC detection is a diagnostic technique providing for visualization of c-Met antigens after their localization with a primary anti-c-Met antibody. In some embodiments, the primary anti-c-Met antibody is selected from a mouse IgG, mouse IgM or a rabbit antibody. In some embodiments, IHC detection comprises direct visualization of a primary anti-cMet antibody. In some embodiments, IHC detection comprises indirect visualization of a primary anti-cMet antibody. In some embodiments, indirect visualization comprises a secondary antibody specific for the species of primary anti-cMet antibody. In some embodiments, indirect visualization further comprises a tertiary antibody that binds to the secondary antibody, wherein the tertiary antibody is conjugated to at least one enzyme. In some embodiments, indirect visualization further comprises a chromogen having a substrate specific for the at least one enzyme of the tertiary antibody. In some embodiments, the chromogen produces a detectable precipitate, preferably wherein the precipitate is detectable by visualization and/or colorimetric shift. In certain embodiments, the substrate is hydrogen peroxide. In certain embodiments, the chromogen is 3, 3′-diaminobenzidine tetrahydrochloride (DAB).

In some embodiments, expression of c-Met is determined using an OptiView DAB IHC Detection Kit (Ventana Catalog Number 760-700). Optiview kits use an indirect method to visualize specific mouse and rabbit primary antibodies bound to an antigen by depositing a brown colored precipitate.

### Preparation of Tissue for IHC Detection

In some embodiments, IHC detection of c-Met expression comprises the step of staining tumor tissue that is frozen, formalin-fixed, and/or paraffin-embedded. In some embodiments, tumor tissue staining is performed via slide staining device. As contemplated, such a slide staining device automates a slide staining step, for example, washing the slide to remove unbound material after an antibody incubation step and/or applying a coverslip to the slide. In some embodiments, the slide staining device is a VENTANA© slide staining device. In some embodiments, the slide staining device is a VENTANA® BenchMark Series instrument (i.e., a BenchMark ULTRA IHC/ISH System).

Formalin-fixed, paraffin-embedded tissues are suitable for use with OptiView DAB IHC Detection Kit and VENTANA® BenchMark Series instruments. In some embodiments, preparation of tumor tissue for IHC detection comprises the step of contacting the tumor tissue with a fixative. In some embodiments, a formalin-based fixative is used, e.g., 10% neutral buffered formalin (NBF).

To minimize variability of visualization results, tumor tissue section thickness, fixation type and duration may be optimized. In some embodiments, tumor tissue sections are of a thickness of about 2 m to about 6 m. In some embodiments, tumor tissue sections are of a thickness of about 2, about 3, about 4, about 5, or about 6 m. Slide heating is contemplated for drying tumor tissue sections after slide mounting, or to enhance tissue adhesion to the glass microscope slides. In some embodiments, a slide containing the tumor tissue section is heated, preferably it is baked. In some embodiments, the slides are heated for between 2 and 24 hours at 60° C.±5° C. Avoid excessive heating of the tumor tissue, as it may decrease antigen availability. In some embodiments, the slides are contacted with cold acetone (i.e., 4-8° C.) for ten minutes. In some embodiments, the slides are air dried for at least 30 minutes following contact with cold acetone, preferably overnight.

### Controls

A positive tissue control is contemplated as being run with the c-Met IHC staining procedure. The positive tissue control can be, for example, a tumor tissue or a non-neoplastic gallbladder tissue. Some or all of a positive tissue control will feature strong staining. A positive tissue control may contain both positive and negative staining and serve as both the positive and negative control tissue. Cellular components that do not stain should demonstrate an absence of specific staining, which provides an indication of background staining. In some embodiments, the same tissue used for the positive tissue control is used as the negative tissue control. In some embodiments, the tissue for the positive or negative control is prepared in a manner identical to the test tissue.

A negative control aids interpretation of c-Met IHC scores. In some embodiments, a negative reagent control is used in place of the primary anti-c-Met antibody to evaluate nonspecific staining. In some embodiments, the negative control reagent is the diluent alone. In preferred embodiments, the incubation period for the negative reagent control equals the incubation period for the primary antibody.

Prior to initial use of a primary anti-cMet antibody in the methods of treatment described herein, the specificity of the antibody must be verified by testing performance on a series of tissues with known immunohistochemistry performance characteristics for c-Met expression, with respect to positive and negative tissues.

### Interpretation of Results

The OptiView DAB IHC Detection Kit (Ventana Catalog Number 760-700) as contemplated for the invention causes a brown colored reaction product to precipitate at or about the c-Met antigen sites localized by the primary anti-c-Met antibody.

In some embodiments, detection of c-Met expression by IHC is performed by a qualified pathologist experienced in immunohistochemical procedures. In some embodiments, detection of c-Met expression occurs after the step of evaluating the positive and negative controls. Staining of negative reagent controls are noted, and these results compared to the stained material to verify that the visualization observed is not due to nonspecific interactions. Positive tissue control is examined to verify proper functioning of reagents. If the positive tissue control fails to demonstrate positive staining, results with the test specimens should be considered invalid for purposes of the methods of treatment disclosed herein. Negative tissue control should be examined after the positive tissue control to verify the specific labeling of the target antigen by the primary antibody. The absence of specific staining in the negative tissue control confirms the lack of anti-c-Met primary antibody binding. If specific staining occurs in the negative tissue control, results with the test specimens should be considered invalid for the purposes of the methods of treatment disclosed herein. Nonspecific staining may have a diffuse appearance. Sporadic light staining may also indicate excessive formalin fixation of cells. Necrotic or degenerated cells may stain nonspecifically.

In some embodiments of the methods of treatment with Teliso-V, intact cells of the NSCLC tissue are assayed for c-Met expression by IHC. Tissue sample biopsies, resections or cytology samples of the patient are examined after controls, as described herein, with staining intensity assessed within the context of any non-specific background staining of the controls (i.e., the negative tissue control, and the negative reagent control.) The morphology of cells of the NSCLC tissue sample should also be examined by a qualified pathologist experienced in immunohistochemical procedures. In some embodiments, the NSCLC tissue sample is contacted with a hematoxylin or eosin stain.

### Automated Slide Staining

In some embodiments, patients having NSCLC selected for treatment with Teliso-V have c-Met overexpression determined for tumor tissue by c-Met IHC assay, wherein the c-Met IHC assay is the c-Met Teliso-V Staining Protocol. In some embodiments, the c-Met IHC assay is performed on an automated slide stainer. In some embodiments, the c-Met IHC assay comprises the steps of: 1) applying a bar code label to a slide having tumor tissue, wherein the bar code corresponds to one or more automated IHC protocols to be performed by an automated slide stainer; 2) loading a primary anti-c-Met antibody, at least one negative reagent control, and/or one or more detection reagents onto the automated slide stainer; 3) loading at least one slide having tumor tissue onto the automated slide stainer; 4) running the automated slide stainer according to the one or more automated IHC protocols, thereby staining c-Met tumor tissue on the slides; and 5) detecting and scoring the c-Met IHC staining of the slides. In some embodiments, the automated slide stainer is a Ventana BenchMark series instrument, optionally a Ventana Benchmark Ultra automated staining instrument. In some embodiments, the bar code corresponds to an SP44 IHC protocol. In some embodiments, the SP44 IHC protocol is selected from a Deparaffinization protocol, a Cell Conditioning protocol (i.e., Ventana Catalog No. 950-224), an Antibody protocol (i.e., for SP44: Ventana Catalog No. 790-4430, or for Rabbit Monoclonal Negative Control Ig: Ventana Catalog No. 790-4795), a Detection protocol (i.e., for OptiView DAB IHC Detection Kit: Ventana Catalog No. 760-700), or a Counterstain protocol (i.e., for hematoxylin II: Ventana Catalog No. 790-2208, or for bluing reagent: Ventana Catalog No. 760-2037). In some embodiments, the primary anti-cMet antibody is the SP44 antibody. In some embodiments the one or more negative reagent controls is a negative control immunoglobulin. In some embodiments, the one or more detection reagents are from an OptiView detection kit, optionally selected from Reaction Buffer (Ventana Catalog No. 950-300), Ultra Liquid Coverslip (Ventana Catalog No. 650-210), or EZ Prep (Ventana Catalog No. 950-102).

### 7. EXAMPLES

The following Examples, which highlight certain features and properties of the exemplary embodiments of the antibodies and binding fragments described herein are provided for purposes of illustration.

### 7.1. Example 1: c-Met Teliso-V Staining Protocol

The following assay was developed to determine the suitability of a candidate patient having NSCLC for selection to be treated with the combination of Teliso V and osimertinib. An IHC staining assay for determining c-Met-overexpression was developed using the Ventana c-Met CONFIRM (SP44) kit and c-Met SP44 OptiView IHC Staining Assay.

This assay and aspects of its protocol are suitable for use in the pre-screening of patients having NSCLC for treatment with telisotuzumab vedotin, or treatment with a biological product having biosimilarity to telisotuzumab vedotin.

### 7.1.1. Materials and Methods

### Specimen Preparation

Routinely processed, formalin fixed, paraffin embedded tumor tissues were thin-sectioned at about 4 microns, and floated onto positively charged glass slides. Tissue was fixed using 10% neutral buffered formalin. Slides were stained immediately after sectioning, to avoid time-dependent reduction of antigenicity.

### Immunohistochemistry Procedure

Immunohistochemistry (IHC) for c-Met was performed on the Ventana BenchMark Ultra automation staining platform. The primary antibody used was the anti-c-Met clone SP44. The OptiView DAB IHC Detection Kit was used for indirect visualization of the primary antibody for c-Met expression determination.

The procedures for staining on the Ventana Benchmark instrument included the steps of:
1) applying a slide bar code label corresponding to the SP44 IHC protocol to be performed (TABLE 3);
2) loading the SP44 antibody, Rabbit Monoclonal Negative Control Ig, and OptiView detection kit dispensers onto the reagent tray; optionally checking bulk fluids (TABLE 4) and empty waste;
3) loading slides to be examined onto the automated slide stainer; and
4) starting the staining run on the Ventana instrument.

At the completion of the Ventana instrument run, slides were removed and submerged in a mild detergent to remove the oil coverslip. Slides were rinsed thoroughly with distilled water, and then dehydrated through graded series of alcohols. Slides were cleared in xylene and a coverslip applied using a permanent mounting media.

TABLE 3
c-Met SP44 OptiView IHC Staining Protocol

| | | VENTANA |
|---|---|---|
| PROCEDURE TYPE | METHOD | CATALOG # |
| Deparaffinization | Selected | N/A |
| Cell Conditioning | Standard Cell | 950-224 |
| | Conditioning 1 | |
| Antibody | SP44 or Rabbit | 790-4430 or 790-4795 |
| | Monoclonal | |
| | Negative Control Ig | |
| | (16 minutes) | |
| Detection | Opti View DAB IHC | 760-700 |
| | Detection Kit | |
| | (8 minutes) | |
| Counterstain | Hematoxylin II | 790-2208 |
| | (4 minutes) | |
| Counterstain | Bluing Reagent | 760-2037 |
| | (4 minutes) | |

TABLE 4
Bulk fluids for c-Met SP44 OptiView Staining Protocol

| BULK REAGENTS | VENTANA CATALOG # |
|---|---|
| Reaction Buffer | 950-300 |
| Ultra Liquid Coverslip | 650-210 |
| EZ Prep | 950-102 |

### 7.1.2. Results and Analysis

### Slide Evaluation and Interpretation

Neoplastic cells stained with the c-MET SP44 OptiView IHC assay were evaluated visually for positivity based on the intensity of the diaminobenzidine (DAB) signal. The IHC signal may be distributed homogeneously throughout the neoplasm or distributed heterogeneously with few cells staining positive.

c-MET (SP44) IHC staining in NSCLC showed that membrane staining is often accompanied by cytoplasmic staining (i.e., both cytoplasmic and membranous). When the staining pattern presents as membranous, it can be either circumferential (the predominant showing), or partial (i.e., basolateral staining in adenocarcinomas). Both membranous and cytoplasmic staining showed a range of intensity varying from no staining (IHC score of 0) to strong staining (IHC score of 3+). Cytoplasmic staining was generally lower in intensity than membranous staining. Some situations showed cytoplasmic staining having a similar intensity to membrane staining (i.e., especially in cases with moderate or strong intensities), and careful differentiation between membranous from cytoplasmic staining was required.

Normal lung, bronchial epithelium, pneumocytes and alveolar macrophages generally did not show strong levels of c-Met-overexpression. However, bronchial epithelium and pneumocytes stained with an IHC score of 2+ to 3+ in a basolateral pattern. Staining in normal cellular components may be suitable for internal controls of the method. Representative staining intensity guidelines for cytoplasmic staining and membranous staining are shown in TABLE 5 and TABLE 6, respectively.

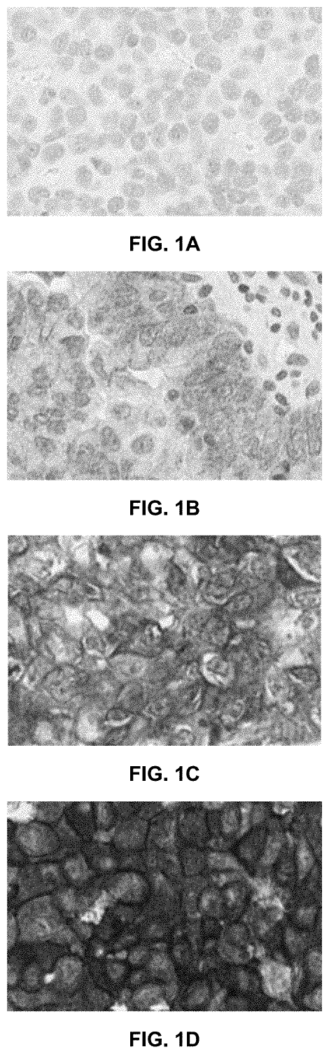

TABLE 5
Cytoplasmic staining intensities
for c-Met on non-squamous NSCLC

| | | Exemplary (20×) |
|---|---|---|
| | | Cytoplasmic |
| | | Staining for |
| Staining Description | Intensity | Non-Sq NSCLC |
| No staining or non-specific | No staining | FIG. 1A |
| background staining | | |
| Pale/light brown or tan in | Weak/1+ | FIG. 1B |
| color above the level of non- | staining | |
| specific background staining. | | |
| Often requires high magnification | | |
| (20× or 40×) to view staining | | |
| Medium brown in color with higher | Moderate/2+ | FIG. 1C |
| intensity than 1+. Often seen at | staining | |
| low magnification (4× or 10×) | | |
| Dark brown often with a black hue. | Strong/3+ | FIG. 1D |
| Easily visualized at low | staining | |
| magnification (2× or 4×) | | |

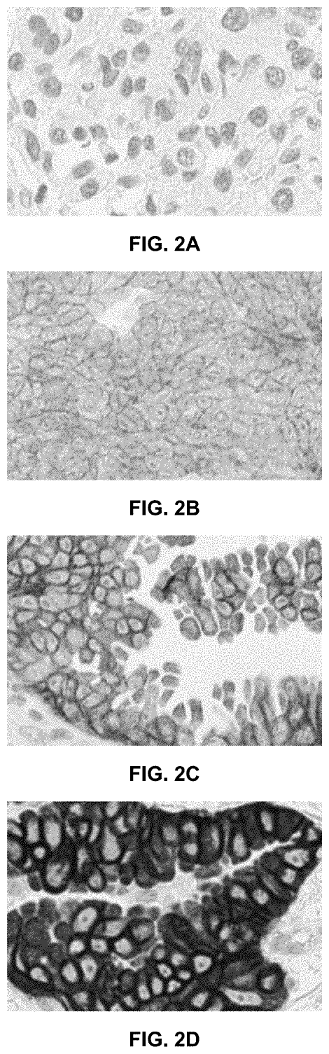

TABLE 6
Membranous staining intensities
for c-Met on non-squamous NSCLC

| | | Exemplary (20×) |
|---|---|---|
| | | Membranous |
| | | Staining for |
| Staining Description | Intensity | Non-Sq NSCLC |
| No staining or non-specific | No staining | FIG. 2A |
| background staining | | |
| Pale/light brown or tan in | Weak/1+ | FIG. 2B |
| color above the level of non- | staining | |
| specific background staining. | | |
| Often requires high magnification | | |
| (20× or 40×) to view staining | | |
| Medium brown in color with higher | Moderate/2+ | FIG. 2C |
| intensity than 1+. Often seen at | staining | |
| low magnification (4× or 10×) | | |
| Dark brown often with a black hue. | Strong/3+ | FIG. 2D |
| Easilyvisualized at low | staining | |
| magnification (2× or 4×) | | |

### Scoring Algorithms

c-Met-stained tumor tissue IHC slides were evaluated for membrane staining on neoplastic cells. Non-squamous NSCLC samples with <25% 3+ membrane staining were considered c-Met negative. Non-squamous NSCLC samples with ≥25% of viable tumor cells exhibiting 3+ membrane staining were considered c-Met positive. Non-squamous NSCLC samples with ≥25% to <50% of viable tumor cells exhibiting 3+ membrane staining were considered c-Met Intermediate. Non-squamous NSCLC samples with ≥50% of viable tumor cells exhibiting 3+ membrane staining were considered c-Met High. TABLE 7 shows different intensities of membrane staining and positive/negative status per scoring algorithm.

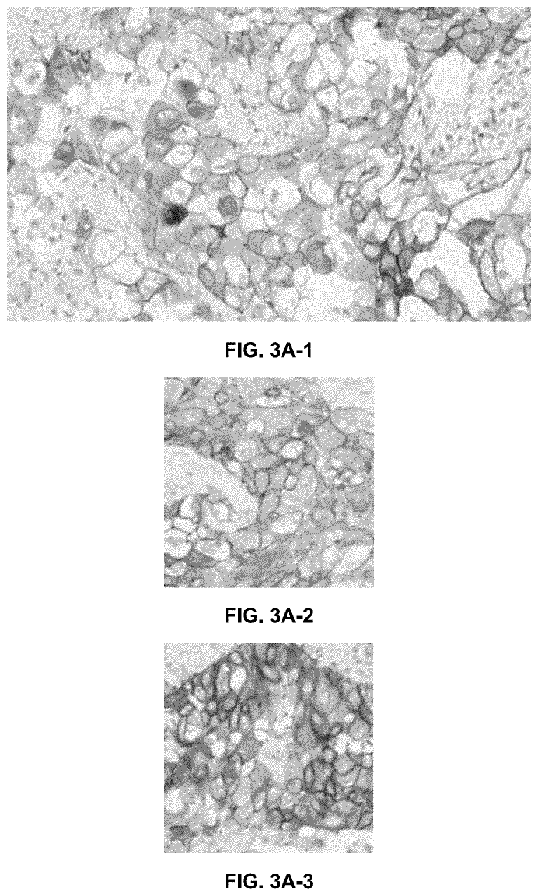

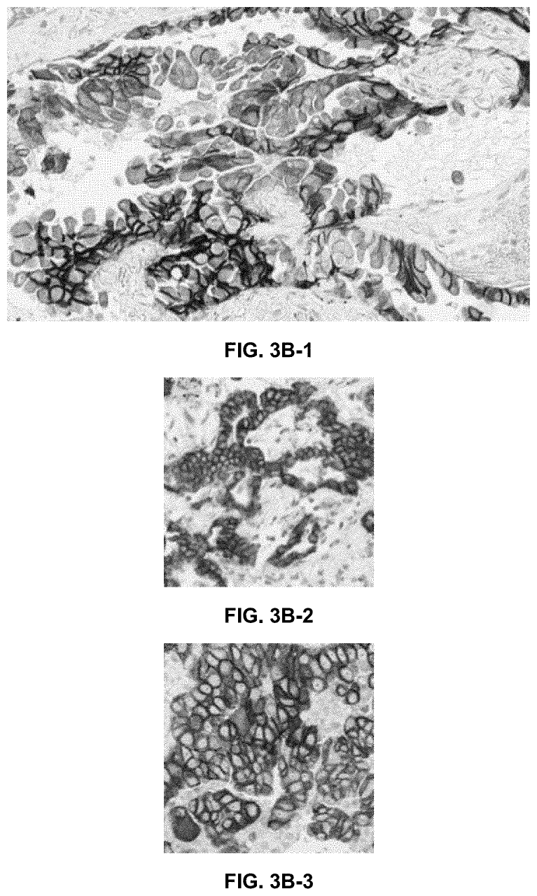

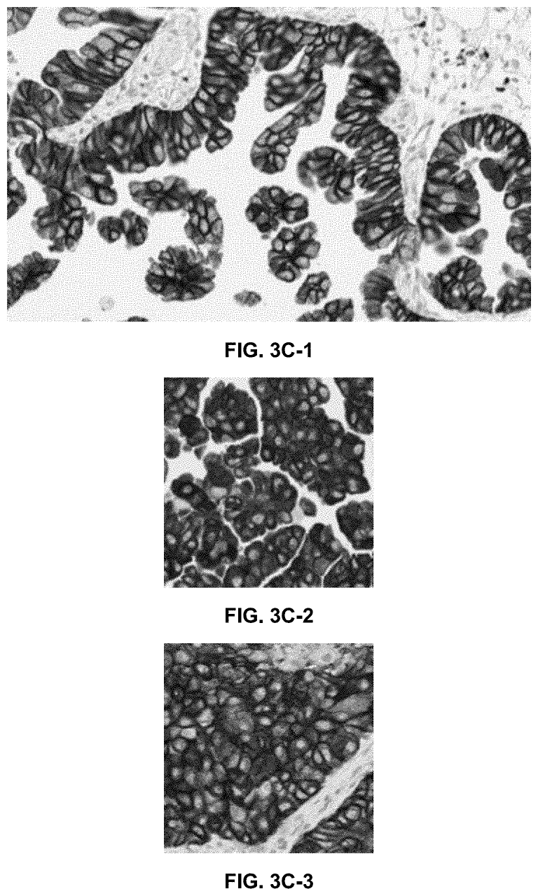

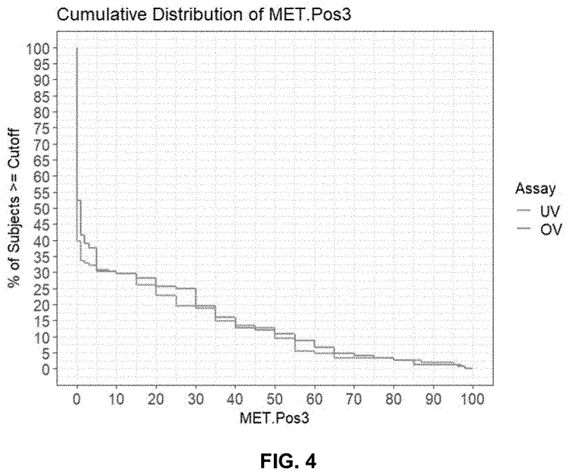

TABLE 7
c-Met Scoring Algorithm - Non-Squamous NSCLC: Representative
cutoffs for c-Met positive and c-Met high

| | | Exemplary (20x) |
|---|---|---|
| c-MET IHC Staining | Interpretation | Representative Images |
| <25% of neoplastic cells from | Negative | FIG. 3A1-3A3 |
| tumor tissue have 3+ | | |
| membrane staining | | |
| ≥25% of neoplastic cells from | c-Met Positive | FIG. 3B1-3B3 |
| tumor tissue have 3+ | | |
| membrane staining | | |
| ≥50% of neoplastic cells from | c-Met High | FIG. 3C1-3C3 |
| tumor tissue have 3+ | | |
| membrane staining | | |

A second scoring algorithm is also utilized. NSCLC samples are assessed for whether they exhibit c-Met overexpression or lack c-Met overexpression. c-Met overexpression is defined according to several different c-Met staining cutoffs, as follows:

TABLE 8
c-Met Scoring Algorithm - Non-Squamous NSCLC: Six Representative Pairs of Cutoffs
for Determining c-Met Overexpression and Lack of c-Met Overexpression

| # | c-Met Overexpression | Lack of c-Met Overexpression |
|---|---|---|
| 1 | ≥25% of neoplastic cells from tumor tissue have | <25% of neoplastic cells from tumor tissue have |
| | ≥1+ membrane and/or cytoplasmic staining | ≥1+ membrane and/or cytoplasmic staining |
| 2 | ≥25% of neoplastic cells from tumor tissue have | <25% of neoplastic cells from tumor tissue have |
| | ≥2+ membrane and/or cytoplasmic staining | ≥2+ membrane and/or cytoplasmic staining |
| 3 | ≥25% of neoplastic cells from tumor tissue have | <25% of neoplastic cells from tumor tissue have |
| | 3+ membrane and/or cytoplasmic staining | 3+ membrane and/or cytoplasmic staining |
| 4 | ≥50% of neoplastic cells from tumor tissue have | <50% of neoplastic cells from tumor tissue have |
| | 3+ membrane and/or cytoplasmic staining | 3+ membrane and/or cytoplasmic staining |

### 7.1.3. Validation Across IHC Platforms

Comparison of c-Met IHC Assays: SP44 “Ultraview” to SP44 “Optiview”

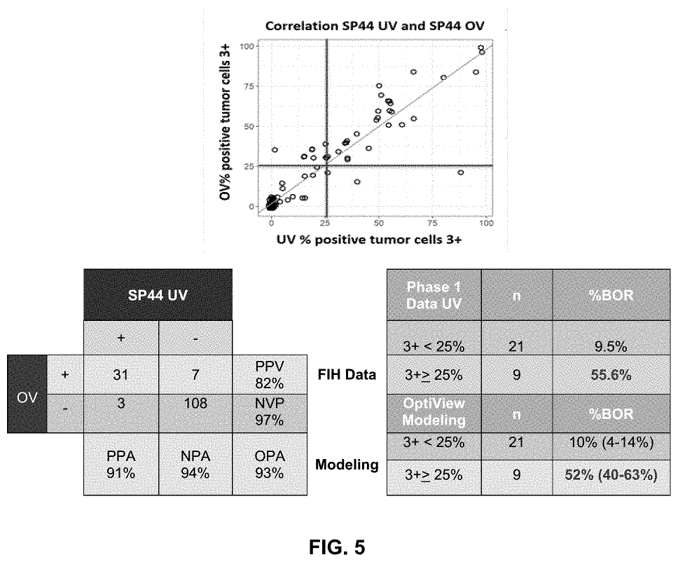

An analytical method comparison between SP44 Ultraview and SP44 OptiView on a large cohort of commercial NSCLC tissues was performed (FIG. 4). These results show that SP44 OptiView IHC assay selected for a similar patient population as SP44 Ultraview at the ≥25% 3+ membrane staining cutoff. The overall percent agreement at this cutoff between the two assays was 93%. The first in human phase I clinical trial for Teliso-V monotherapy in NSCLC enrolled patients with an H-score of 150 using the SP44 UltraView assay. Upon re-scoring of the slides, the optimal cutoff utilizing the SP44 Ultraview IHC assay was ≥25% 3+ with a best overall response of 56%. The best overall response for the OptiView IHC assay at the same cutoff, ≥25% 3+, utilizing the phase I data through statistical modeling was 52% (FIG. 5).

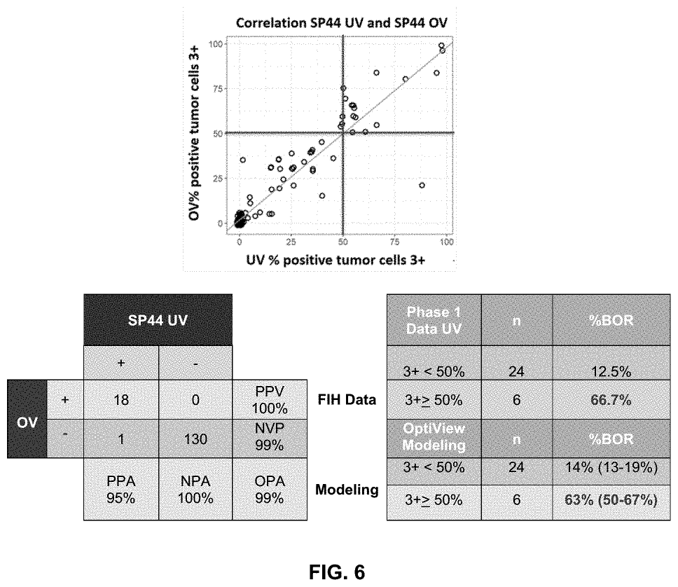

Similarly, the SP44 OptiView IHC assay showed a 99% overall percent agreement to the SP44 Ultraview IHC assay at the ≥50% 3+ membrane staining cutoff (FIG. 6). The first in human phase I clinical trial for Teliso-V monotherapy in NSCLC enrolled patients with an H-score of 150 using the SP44 UltraView assay. Upon re-scoring of the slides, the SP44 Ultraview IHC assay showed a best overall response of 67% at the ≥50% 3+ membrane staining cutoff. The best overall response for the OptiView IHC assay at the same cutoff, ≥50% 3+, utilizing the phase I data through statistical modeling was 63% (FIG. 6).

### 7.2. Example 2: Results of Multicenter, Phase 1/1b Study of ABBV-399 in Combination with Osimertinib in Subjects with Advanced Solid Tumors

### 7.2.1. Summary

An ongoing Phase 1/1b open-label study (clinicaltrials.gov Identifier NCT02099058, incorporated by reference) is evaluating the safety, pharmacokinetics (PK), and preliminary efficacy of ABBV-399 (Teliso-V) as monotherapy and in combination with, inter alia, osimertinib in participants with advanced solid tumors likely to express c-Met..

The subjects treated with the combination of Teliso-V and osimertinib had the following characteristics:
c-Met+ locally advanced or metastatic NSCLC with c-Met overexpression per designated IHC laboratory assay specification using a c-Met positive cutoff of ≥25% 3+ membrane staining in a tumor sample.
NSCLC with EGFR mutations del19 or L858R, with or without T790M mutation, and none of the EGFR mutations known to be resistant to osimertinib.
At least 1 but no more than 2 prior treatment regimens, one of which must have contained osimertinib. Consecutive EGFR tyrosine kinase inhibitor treatments are counted as 1 regimen.
Disease progression while on prior osimertinib treatment.
Only 1 prior regimen may have contained chemotherapy.

Teliso V was administered via intravenous (IV) infusion over a 30±10 minutes period at either 1.6 mg/kg or 1.9 mg/kg every two weeks, and osimertinib was administered orally at 80 mg daily. The overall response rate ORR of the combination of Teliso-V and osimertinib was determined per Investigator assessment. The ORR was defined as the proportion of subjects with a confirmed complete response (CR) or confirmed partial response (PR) based on RECIST, version 1.1. Tumor assessments were performed at baseline and every 8 weeks according to RECIST v1.1.

### Safety

Safety and tolerability were assessed by evaluating adverse events (AEs) and changes in laboratory data and vital signs for the entire study duration. AE severity was graded according to the National Cancer Institute Common Terminology Criteria for Adverse Events v4.03. Treatment-emergent AEs (TEAEs) were those that occurred during treatment or up to 60 days after discontinuation of telisotuzumab vedotin.

Safety analyses included all patients who received ≥1 dose of the combination of Teliso-V and osimertinib. Efficacy analyses included patients—who received ≥1 dose of Teliso-V and osimertinib and ≥1 post-baseline tumor assessment (or had clinical evidence of progression or died prior to post-baseline assessment).

### RECIST (Version 1.1) Criteria for Tumor Response

Response criteria were assessed using RECIST (version 1.1). Changes in the measurable lesions over the course of therapy were evaluated using the criteria listed below.

a. Eligibility

Subjects with measurable disease at Baseline can have objective tumor response evaluated by RECIST criteria. Measurable disease is defined by the presence of at least one measurable lesion. If the measurable disease is restricted to a solitary lesion, its neoplastic nature should be confirmed by cytology/histology if possible.

b. Measurability

TABLE 9
Measurability factors

| | |
|---|---|
| Measurable | Lesions accurately measured in at least one |
| Lesions | dimension with a minimum size of: |
| | longest diameter ≥10 mm (CT scan slice |
| | thickness no greater than 5 mm) |
| | 10 mm caliper measurement by clinical exam |
| Non-Measurable | All other lesions, including small lesions (longest |
| Lesions | diameter <10 mm) as well as truly non-measurable |
| | lesions. Lesions considered truly non-measurable |
| | include: leptomeningeal disease, ascites, |
| | pleural/pericardial effusion, inflammatory breast |
| | disease, lymphangitic involvement of skin or lung |
| | and also abdominal masses that are not confirmed |
| | and followed by imaging techniques. |
| Measurable | To be considered pathologically enlarged and |
| Malignant | measurable, a lymph node must be ≥15 mm in |
| Lymph Nodes | short axis when assessed by CT scan (CT scan |
| | slice thickness recommended to be no greater |
| | than 5 mm). At baseline and in follow-up, only |
| | the short axis will be measured and followed. |
| Non-Measurable | Pathological lymph nodes with ≥10 to <15 mm |
| Malignant | short axis. |
| Lymph Nodes | |

All measurements should be taken and recorded in metric notation, using calipers if clinically assessed. All baseline evaluations should be performed as closely as possible to the beginning of treatment and not more than 4 weeks before the beginning of the treatment.

The same method of assessment and the same technique should be used to characterize each identified and reported lesion at Baseline and during follow-up.

Clinical lesions will only be considered measurable when they are superficial (e.g., skin nodules and palpable lymph nodes) and >10 mm diameter as assessed using calipers. For the case of skin lesions, documentation by color photography including a ruler to estimate the size of the lesion is recommended.

c. Methods of Measurement

Conventional CT should be performed with cuts of 5 mm or less in slice thickness contiguously. This applies to tumors of the chest and abdomen. A scale should be incorporated into all radiographic measurements.

Cytology and histology can be used to differentiate between partial response (PR) and complete response (CR), when needed in rare cases.

d. Baseline Documentation of “Target” and “Non-Target” Lesions

All measurable lesions up to a maximum of 2 lesions per organ and 5 lesions in total, representative of all involved organs were identified as target lesions and recorded and measured at Baseline. Tumor lesions situated in a previously irradiated area, or in an area subjected to other loco-regional therapy, are usually not considered measurable unless there has been demonstrated progression in the lesion.

Lymph nodes merit special mention since they are normal anatomical structures which may be visible by imaging even if not involved by tumor. Pathological nodes which are defined as measurable and may be identified as target lesions must meet the criterion of a short axis of ≥15 mm by CT scan. Only the short axis of these nodes will contribute to the baseline sum. The short axis of the node is the diameter normally used by radiologists to judge if a node is involved by solid tumor. Nodal size is normally reported as two dimensions in the plane in which the image is obtained (for CT scan this is almost always the axial plane). The smaller of these measures is the short axis. For example, an abdominal node which is reported as being 20 mm×30 mm has a short axis of 20 mm and qualifies as a malignant, measurable node. In this example, 20 mm should be recorded as the node measurement. All other pathological nodes (those with short axis ≥10 mm but <15 mm) should be considered non-target lesions. Nodes that have a short axis <10 mm are considered non-pathological and should not be recorded or followed.

A sum of diameters for all target lesions were calculated and reported as the baseline sum of diameters. If lymph nodes were to be included in the sum, then as noted above, only the short axis were added into the sum. The baseline sum diameters were used as a reference by which to characterize the objective tumor response.

All other lesions (or sites of disease) including pathological lymph nodes should be identified as non-target lesions and should also be recorded at Baseline. Measurements of these lesions are not required, but the presence (stable, increasing or decreasing) or absence of each should be noted throughout follow-up.

e. Evaluation of Target Lesions

### Complete Response (CR):

The disappearance of all target lesions. Any pathological lymph nodes (whether target or non-target) must have reduction in short axis to <10 mm.

### Partial Response (PR):

At least a 30% decrease in the sum of diameters of target lesions, taking as reference the baseline sum diameters.

### Progressive Disease (PD):

At least a 20% increase in the sum of the diameters of target lesions, taking as reference the smallest sum of diameters recorded since the treatment started (baseline or after) or the appearance of one or more new lesions. In addition to the relative increase of 20%, the sum must also demonstrate an absolute increase of at least 5 mm.

### Stable Disease (SD):

Neither sufficient shrinkage to qualify for PR nor sufficient increase to qualify for PD, taking as reference the smallest sum of diameters since the treatment started (baseline or after).

### Assessment of Target Lesions:

Lymph nodes identified as target lesions should always have the actual short axis measurement recorded (measured in the same anatomical plane as the baseline examination), even if the nodes regress to below 10 mm on study. This means that when lymph nodes are included as target lesions, the ‘sum’ of lesions may not be zero even if complete response criteria are met, since a normal lymph node is defined as having a short axis of <10 mm. For PR, SD and PD, the actual short axis measurement of the nodes is to be included in the sum of target lesions.

All lesions (nodal and non-nodal) recorded at Baseline should have their actual measurements recorded at each subsequent evaluation, even when very small (<5 mm). However, sometimes target lesions or lymph nodes become too small to measure. If it is in the opinion of the radiologist that the lesion has likely disappeared, the measurement should be recorded as 0 mm. If the lesion is believed to be present, but too small to measure, a default value of 5 mm should be assigned (as derived from the 5 mm CT slice thickness). The measurement of these lesions is potentially non-reproducible; therefore, providing this default value will prevent false responses or progression based upon measurement error.

f. Evaluation of Non-Target Lesions

### Complete Response (CR):

The disappearance of all non-target lesions and normalization of tumor marker level. All lymph nodes must be non-pathological in size (<10 mm short axis).

### Non-CR/Non-PD:

Persistence of one or more non-target lesion(s) and/or maintenance of tumor marker level above the normal limits.

### Progressive Disease (PD):

Unequivocal progression of existing non-target lesions.

In this setting, to achieve ‘unequivocal progression’ on the basis of non-target disease, there must be an overall level of substantial worsening in non-target disease such that, even in the presence of SD or PR in target disease, the overall tumor burden has increased sufficiently to merit discontinuation of therapy. A modest ‘increase’ in the size of one or more non-target lesions is usually not sufficient to qualify for unequivocal progression status. The designation of overall progression solely on the basis of change in non-target disease in the face of SD or PR of target disease will therefore be extremely rare.

Note: If the subject discontinues treatment for symptomatic deterioration, every effort should be made to document objective progression even after discontinuation of treatment.

### New Lesions

The appearance of new malignant lesions denotes disease progression. While there are no specific criteria for the identification of new radiographic lesions, the findings of a new lesion should be unequivocal, i.e., not attributable to differences in scanning technique, timing of scanning, phase of contrast administration, change in imaging modality or finding thought to represent something other than tumor (e.g., some ‘new’ bone lesions may be simply healing or flare of pre-existing lesions). A lesion identified on a follow-up study in an anatomical location that was not scanned at Baseline is considered a new lesion and will indicate disease progression. An example of this is the subject who has visceral disease at Baseline and while on study has a CT or MRI brain ordered which reveals metastases. The subject's brain metastases are considered evidence of progressive disease even if he/she did not have brain imaging at Baseline.

If a new lesion is equivocal (i.e., too small to measure), continued therapy and follow-up evaluation should clarify if it represents truly new disease. If repeat scans confirm there is a new lesion, then progression should be declared using the date of the initial scan.

### 7.2.2. Results

In an interim analysis, treatment of non-squamous NSCLC EGFR mutant subjects who had been previously treated with osimertinib and experienced progressive disease while on osimertinib with the combination of 1.9 mg/kg Teliso-V once every two weeks and 80 mg osimertinib daily resulted in an ORR of 66.7%. In a subsequent interim analysis including more subjects, the ORR was 53%. The combination of 1.6 mg/kg Teliso-V once every two weeks and 80 mg osimertinib daily resulted in an ORR of 42.9%. Together, the two treatment groups experienced an ORR of 50%.

### 7.3. Discussion

Teliso-V at a dose of 1.6 or 1.9 mg/kg every 2 weeks in combination with 80 mg osimertinib daily demonstrated a promising ORR and tolerable safety profile in the non-squamous EGFR mutant NSCLC cohort with c-Met overexpression.

Results from an interim analysis of an ongoing Phase 2 study of Teliso-V monotherapy at a dose of 1.9 mg/kg (ClinicalTrials.gov Identifier: NCT03539536) in c-Met positive (≥25% 3+IHC staining) advanced non-squamous NSCLC subjects demonstrated an ORR (defined as either complete response or partial response) in non-squamous NSCLC EGFR mutant patients of 11.6%. In the subset of those non-squamous NSCLC EGFR mutant subjects who had been previously treated with osimertinib, the ORR was 0%. In contrast, in the Phase 1 study of Teliso-V in combination with osimertinib (ClinicalTrials.gov Identifier: NCT02099058), treatment of non-squamous NSCLC EGFR mutant subjects who had been previously treated with osimertinib and experienced progressive disease while on osimertinib with the combination of 1.9 mg/kg Teliso-V once every two weeks and 80 mg osimertinib daily initially resulted in an ORR of 66.7%. As the trial proceeded and more subjects were included in the analysis, the ORR for this group was 53%. The combination of 1.6 mg/kg Teliso-V once every two weeks and 80 mg osimertinib daily resulted in an ORR of 42.9%. Combining the results from the group that received administration of 1.6 mg/kg plus osimertinib and the group that received 1.9 mg/kg osimertinib, the ORR was 50%. These results are summarized in the following table:

TABLE 10
Efficacy of Teliso-V Alone and Teliso-V +
Osimertinib in NSCLC EGFR MU

| Treatment | ORR (CR + PR)^{a} |
|---|---|
| (NSCLC EGFR MU | by ICR, n/N (%) |
| patient sub-population) | [95% CI] |
| Teliso-V at 1.9 mg/kgb | 11.6% |
| | [3.9%, 25.1%] |
| Teliso V at 1.9 mg/kg | 0% |
| (Prior osimertinib therapy) | |
| Teliso V at 1.6 mg/kg + osimertinib | 42.9% |
| (Progressive disease on prior | [9.9%, 81.6%] |
| osimertinib therapy) | |
| Teliso V at 1.9 mg/kg + osimertinib | 66.7% |
| (Progressive disease on prior | [34.9%, 90.1%] |
| osimertinib therapy) | |
| Initial data analysis | |
| Teliso V at 1.9 mg/kg + osimertinib | 53% |
| (Progressive disease on prior | [28%, 77%] |
| osimertinib therapy) | |
| Subsequent data analysis | |

^{a}Confirmed CR or PR per RECIST v1.1.
^{b}c-Met ≥25% 3+ IHC staining, advanced non-squamous NSCLC subjects previously treated with platinum-based chemotherapy and immune checkpoint inhibitor (or prior standard TKI therapy, 2 and 3rd line), results from clinicaltrials.gov Identifier NCT03539536
CI, confidence interval;
CR, complete response;
ICR, independent central review;
MU, mutant;
NSCLC, non-small cell lung cancer;
ORR, objective response rate;
PR, partial response;
RECIST, Response Evaluation Criteria in Solid Tumors

Thus, the administration of two therapeutics, neither of which is effective individually in the NSCLC EGFR mutant patient population previously treated with osimertinib therapy, provides an effective treatment in that population.

### 8. Exemplary Embodiments

While various specific embodiments have been illustrated and described, and some are represented below, it will be appreciated that various changes can be made without departing from the spirit and scope of the inventions(s).
1. A method of treating a non-squamous non-small cell lung cancer (“NSCLC”) tumor that expresses c-Met, comprising administering to a human subject having said NSCLC tumor, wherein the human subject received previous osimertinib therapy and experienced progressive disease while on the osimertinib therapy
   1) osimertinib, and
   2) pharmaceutical composition comprising Teliso-V, an anti-c-Met antibody drug conjugate (“ADC”), wherein the drug conjugate is monomethyl auristatin E (“MMAE”), and the ADC has the following structure:

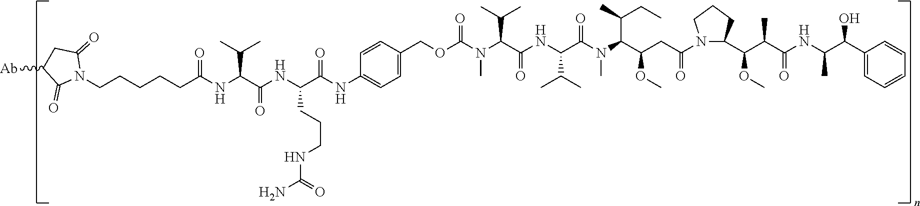

wherein Ab is an IgG antibody consisting of heavy chains each consisting of the amino acid sequence of SEQ ID NO:5 and light chains each consisting of the amino acid sequence of SEQ ID NO: 10, n has a value of 2 or 4, and attachment to the Ab is via a thioether linkage formed with a sulfhydryl group of a cysteine residue, and, wherein ≥25% of neoplastic cells from tumor tissue of the c-Met expressing non-squamous NSCLC from the subject have ≥1+ membrane and/or cytoplasmic staining when assessed by c-Met immunohistochemistry (IHC), wherein the NSCLC tumor carries a mutated EGFR gene.
2. The method of embodiment 1, wherein the mutated EGFR gene comprises an exon 19 deletion or an exon 21 L858R mutation.
3. The method of embodiment 1-2, wherein the mutated EGFR gene comprises a T790M mutation.
4. The method of any one of embodiments 1-3, wherein ≥25% of tumor cells from tumor tissue of the c-Met expressing non-squamous NSCLC from the subject have ≥2+ membrane and/or cytoplasmic staining when assessed by c-Met IHC.
5. The method of any one of embodiments 1-3, wherein ≥25% of tumor cells from tumor tissue of the c-Met expressing non-squamous NSCLC from the subject have 3+ membrane and/or cytoplasmic staining when assessed by c-Met IHC.
6. The method of any one of embodiments 1-3, wherein ≥50% of tumor cells from tumor tissue of the c-Met expressing non-squamous NSCLC from the subject have 3+ membrane and/or cytoplasmic staining when assessed by c-Met IHC.
7. The method of any one of embodiments 1-6, wherein administration of osimertinib and Teliso-V achieves an objective response rate (ORR) in the subject that is greater than 25%, greater than 30%, greater than 35%, greater than 40%, greater than 45%, greater than 50%, greater than 55%, greater than 60%, greater than 65%, or greater than 70% or more.
8. The method of any one of embodiments 1-7, wherein administration of osimertinib and Teliso-V achieves a median duration of response (DoR) in the subject of at least 4 months, at least 6 months, at least 8 months, or at least 10 months.
9. The method of any one of embodiments 1-8, wherein administration of osimertinib and Teliso-V achieves progression free survival (PFS) in the subject of at least 5 months, at least 5.5 months, at least 6.5 months or at least 7.5 months.
10. The method of any one of embodiments 1-9, wherein administration of osimertinib and Teliso-V achieves an overall survival (OS) in the subject of at least 16 months, or an OS of at least 19 months.
11. The method of any one of embodiments 1-9, wherein administration of osimertinib and Teliso-V achieves a partial response (PR) in the subject.
12. The method of any one of embodiments 1-9, wherein administration of osimertinib and Teliso-V achieves a complete response (CR) in the subject.
13. The method of embodiment 1, wherein administration of osimertinib and Teliso-V achieves an objective response rate (ORR) in the subject that is greater than 25%, preferably greater than 35%, and a median duration of response (DoR) in the subject of at least 4 months.
14. The method of any one of embodiments 1-13, wherein the pharmaceutical composition comprising Teliso-V has about a 1:1 ratio of E2 and E4.
15. The method of any one of embodiments 1-14, wherein the pharmaceutical composition comprising Teliso-V has a drug antibody ratio (“DAR”) of about 2.4 to about 3.6.
16. The method of any one of embodiments 1-15, wherein 1.6 or 1.9 mg/kg of Teliso-V is administered intravenously once every two weeks, and 80 mg of osimertinib is administered orally once per day.
17. The method of any one of embodiments 1-15, wherein 1.6 or 1.9 mg/kg of Teliso-V is administered intravenously once every two weeks to subjects weighing 100 kg or less, and 190 mg is administered intravenously to subjects weighing over 100 kg.
18. The method of any one of embodiments 1-17, wherein the tumor tissue is taken prior to administration of the first dose of Teliso-V.
19. The method of any one of embodiments 1-18, wherein the subject has received prior systemic therapy in the locally advanced or metastatic setting.
20. The method of any one of embodiments 1-19, wherein the c-Met IHC is performed according to the c-Met Teliso-V Staining Protocol.
21. A method of treating a non-squamous non-small cell lung cancer (“NSCLC”) tumor that overexpresses c-Met in a human subject, wherein the subject received previous osimertinib therapy and experienced progressive disease while on the osimertinib therapy, comprising determining whether the tumor exhibits c-Met overexpression, wherein c-Met overexpression is defined by ≥25% of the neoplastic cells from tumor tissue of the non-squamous NSCLC having ≥1+ membrane and/or cytoplasmic staining when assessed by IHC; and if the tumor tissue exhibits c-Met overexpression, administering to the subject having said NSCLC tumor 1) osimertinib and 2) a pharmaceutical composition comprising Teliso-V, an anti-c-Met antibody drug conjugate (“ADC”), wherein the drug conjugate is monomethyl auristatin E (“MMAE”), and the ADC has the following structure:

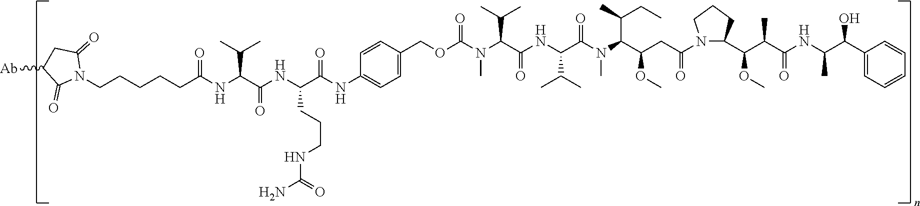

wherein Ab is an IgG antibody consisting of heavy chains each consisting of the amino acid sequence of SEQ ID NO:5 and light chains each consisting of the amino acid sequence of SEQ ID NO: 10, n has a value of 2 or 4, and attachment to the Ab is via a thioether linkage formed with a sulfhydryl group of a cysteine residue wherein the non-squamous NSCLC carries a mutated EGFR gene.
22. The method of embodiment 21, wherein the mutated EGFR gene comprises an exon 19 deletion or an exon 21 L858R mutation.
23. The method of embodiment 22, wherein the mutated EGFR gene comprises a T790M mutation.
24. The method of any one of embodiments 21-23, wherein ≥25% of tumor cells from tumor tissue of the c-Met expressing non-squamous NSCLC from the subject have ≥2+ membrane and/or cytoplasmic staining when assessed by c-Met IHC.
25. The method of any one of embodiments 21-23, wherein ≥25% of tumor cells from tumor tissue of the c-Met expressing non-squamous NSCLC from the subject have 3+ membrane and/or cytoplasmic staining when assessed by c-Met IHC.
26. The method of any one of embodiments 21-23, wherein ≥50% of tumor cells from tumor tissue of the c-Met expressing non-squamous NSCLC from the subject have 3+ membrane and/or cytoplasmic staining when assessed by c-Met IHC.
27. The method of any one of embodiments 21-26, wherein administration of osimertinib and Teliso-V achieves an objective response rate (ORR) in the subject that is greater than 25%, greater than 30%, greater than 35%, greater than 40%, greater than 45%, greater than 50%, greater than 55%, greater than 60%, greater than 65%, or greater than 70% or more.
28. The method of any one of embodiments 21-27, wherein administration of osimertinib and Teliso-V achieves a median duration of response (DoR) in the subject of at least 4 months, at least 6 months, at least 8 months, or at least 10 months.
29. The method of any one of embodiments 21-28, wherein administration of osimertinib and Teliso-V achieves progression free survival (PFS) in the subject of at least 5 months, at least 5.5 months, at least 6.5 months or at least 7.5 months.
30. The method of any one of embodiments 21-29, wherein administration of osimertinib and Teliso-V achieves an overall survival (OS) in the subject of at least 16 months, or an OS of at least 19 months.
31. The method of any one of embodiments 21-29, wherein administration of osimertinib and Teliso-V achieves a partial response (PR) in the subject.
32. The method of any one of embodiments 21-29, wherein administration of osimertinib and Teliso-V achieves a complete response (CR) in the subject.
33. The method of embodiment 21, wherein administration of osimertinib and Teliso-V achieves an objective response rate (ORR) in the subject that is greater than 25%, preferably greater than 35%, and a median duration of response (DoR) in the subject of at least 4 months.
34. The method of any one of embodiments 21-33, wherein the pharmaceutical composition comprising Teliso-V has about a 1:1 ratio of E2 and E4.
35. The method of any one of embodiments 21-34, wherein the pharmaceutical composition comprising Teliso-V has a drug antibody ratio (“DAR”) of about 2.4 to about 3.6.
36. The method of any one of embodiments 21-35, wherein 1.6 or 1.9 mg/kg of Teliso-V is administered intravenously once every two weeks, and 80 mg of osimertinib is administered orally once per day.
37. The method of any one of embodiments 21-35, wherein 1.6 or 1.9 mg/kg of Teliso-V is administered intravenously once every two weeks to subjects weighing 100 kg or less, and 190 mg is administered intravenously to subjects weighing over 100 kg.
38. The method of any one of embodiments 21-37, wherein the tumor tissue is taken prior to administration of the first dose of Teliso-V.
39. The method of any one of embodiments 21-38, wherein the subject has received prior systemic therapy in the locally advanced or metastatic setting.
40. The method of any one of embodiments 21-39, wherein the c-Met IHC is performed according to the c-Met Teliso-V Staining Protocol.
41. The method of embodiment 21, wherein the mutated EGFR gene comprises an exon 19 deletion or an exon 21 L858R mutation, and wherein administration of osimertinib and Teliso-V achieves an objective response rate (ORR) in the subject that is greater than 25%, preferably greater than 35%, optionally wherein administration of osimertinib and Teliso-V achieves a median duration of response (DoR) in the subject of at least 4 months.
42. The method of embodiment 21, wherein 80 mg of osimertinib is administered orally once per day, and 1.6 or 1.9 mg/kg of Teliso-V is administered intravenously once every two weeks, and the pharmaceutical composition comprising the Teliso-V has a drug antibody ratio (“DAR”) of about 2.4 to about 3.6.
43. A method of treating non-squamous non-small cell lung cancer (“NSCLC”) tumors that express c-Met in a plurality of human subjects wherein the human subjects received previous osimertinib therapy and experienced progressive disease while on the osimertinib therapy, comprising the steps of:
   (a) determining whether the tumor exhibits: i) c-Met overexpression or ii) lack of c-Met overexpression, wherein
      i) c-Met overexpression is defined by >25% of the neoplastic cells from tumor tissue of the non-squamous NSCLC having >2+ membrane and/or cytoplasmic staining when assessed by IHC and lack of c-Met overexpression is defined by <25% of the neoplastic cells from tumor tissue of the non-squamous NSCLC having ≥1+ membrane and/or cytoplasmic staining when assessed by IHC;
   (b) if the tumor tissue exhibits lack of c-Met overexpression, excluding the subject having the tumor that exhibits lack of c-Met overexpression from treatment;
   (c) if the tumor tissue exhibits c-Met overexpression, selecting the subject having the tumor that exhibits c-Met overexpression for treatment and administering to the selected subject 1) osimertinib and 2) a pharmaceutical composition comprising Teliso-V, an anti-c-Met antibody drug conjugate (“ADC”), wherein the drug conjugate is monomethyl auristatin E (“MMAE”), and the ADC has the following structure:

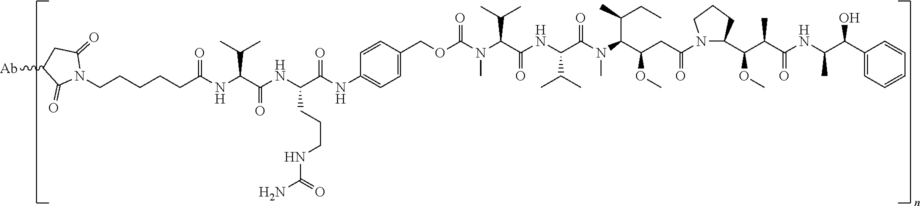

wherein Ab is an IgG antibody consisting of heavy chains each consisting of the amino acid sequence of SEQ ID NO:5 and light chains each consisting of the amino acid sequence of SEQ ID NO: 10, n has a value of 2 or 4, and attachment to the Ab is via a thioether linkage formed with a sulfhydryl group of a cysteine residue, wherein the non-squamous NSCLC tumor carries a mutated EGFR gene.
44. The method of embodiment 43, wherein the mutated EGFR gene comprises an exon 19 deletion or an exon 21 L858R mutation, and wherein administration of osimertinib and Teliso-V achieves an objective response rate (ORR) in the selected subject that is greater than 30% and a median duration of response (DoR) in the subject of at least 4 months.
45. The method of embodiment 43, wherein 80 mg of osimertinib is administered orally once per day, and 1.6 or 1.9 mg/kg of Teliso-V is administered intravenously once every two weeks, and the pharmaceutical composition comprising Teliso-V has a drug antibody ratio (“DAR”) of about 2.4 to about 3.6.
46. The method of any one of embodiments 43-45, wherein the c-Met IHC is performed according to the c-Met Teliso-V Staining Protocol.
47. A method of treating a non-squamous non-small cell lung cancer (“NSCLC”) tumor that expresses c-Met in a human subject, wherein the human subject received previous osimertinib therapy and experienced progressive disease while on the osimertinib therapy, comprising the steps of:
   (a) determining whether the tumor exhibits: i) c-Met overexpression or ii) lack of c-Met overexpression, wherein
      i) c-Met overexpression is defined by ≥25% of the neoplastic cells from tumor tissue of the non-squamous NSCLC having ≥2+ membrane and/or cytoplasmic staining when assessed by IHC and lack of c-Met overexpression is defined by <25% of the neoplastic cells from tumor tissue of the non-squamous NSCLC having ≥1+ membrane and/or cytoplasmic staining when assessed by IHC; or
   (b) if the tumor tissue exhibits c-Met overexpression, administering to the subject 1) osimertinib and 2) a pharmaceutical composition comprising Teliso-V, an anti-c-Met antibody drug conjugate (“ADC”), wherein the drug conjugate is monomethyl auristatin E (“MMAE”), and the ADC has the following structure:

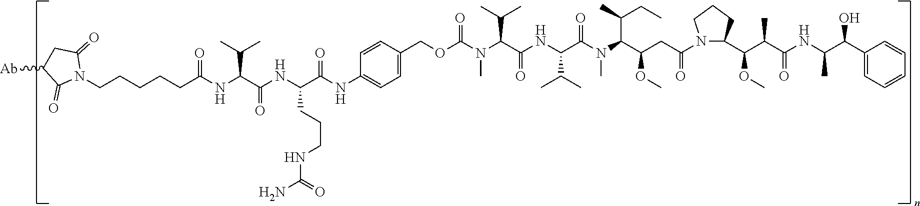

wherein Ab is an IgG antibody consisting of heavy chains each consisting of the amino acid sequence of SEQ ID NO:5 and light chains each consisting of the amino acid sequence of SEQ ID NO: 10, n has a value of 2 or 4, and attachment to the Ab is via a thioether linkage formed with a sulfhydryl group of a cysteine residue, wherein the non-squamous NSCLC tumor carries a mutated EGFR gene.
48. The method of embodiment 47, wherein the mutated EGFR gene comprises an exon 19 deletion or an exon 21 L858R mutation, and wherein administration of osimertinib and Teliso-V achieves an objective response rate (ORR) in the selected subject that is greater than 25% and a median duration of response (DoR) in the subject of at least 4 months.
49. The method of embodiment 47, wherein 80 mg of osimertinib is administered orally once per day, and 1.6 or 1.9 mg/kg of Teliso-V is administered intravenously once every two weeks, and the pharmaceutical composition comprising Teliso-V has a drug antibody ratio (“DAR”) of about 2.4 to about 3.6.
50. A method of treating a non-squamous non-small cell lung cancer (“NSCLC”) tumor that expresses c-Met, comprising administering to a human subject having said NSCLC tumor,
   1) osimertinib, and
   2) pharmaceutical composition comprising Teliso-V, an anti-c-Met antibody drug conjugate (“ADC”), wherein the drug conjugate is monomethyl auristatin E (“MMAE”), and the ADC has the following structure:

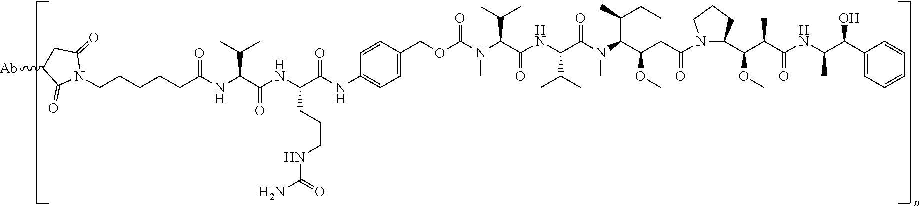

wherein Ab is an IgG antibody consisting of heavy chains each consisting of the amino acid sequence of SEQ ID NO:5 and light chains each consisting of the amino acid sequence of SEQ ID NO: 10, n has a value of 2 or 4, and attachment to the Ab is via a thioether linkage formed with a sulfhydryl group of a cysteine residue, and, wherein ≥25% of neoplastic cells from tumor tissue of the c-Met expressing non-squamous NSCLC from the subject have ≥1+ membrane and/or cytoplasmic staining when assessed by c-Met immunohistochemistry (IHC), wherein the NSCLC tumor carries a mutated EGFR gene.
51. The method of embodiment 50, wherein the mutated EGFR gene comprises an exon 19 deletion or an exon 21 L858R mutation.
52. The method of embodiment 50-51, wherein the mutated EGFR gene comprises a T790M mutation.
53. The method of any one of embodiments 50-52, wherein ≥25% of tumor cells from tumor tissue of the c-Met expressing non-squamous NSCLC from the subject have ≥2+ membrane and/or cytoplasmic staining when assessed by c-Met IHC.
54. The method of any one of embodiments 50-52, wherein ≥25% of tumor cells from tumor tissue of the c-Met expressing non-squamous NSCLC from the subject have 3+ membrane and/or cytoplasmic staining when assessed by c-Met IHC.
55. The method of any one of embodiments 50-52, wherein ≥50% of tumor cells from tumor tissue of the c-Met expressing non-squamous NSCLC from the subject have 3+ membrane and/or cytoplasmic staining when assessed by c-Met IHC.
56. The method of any one of embodiments 50-55, wherein 1.6 or 1.9 mg/kg of Teliso-V is administered intravenously once every two weeks, and 80 mg of osimertinib is administered orally once per day.
57. The method of any one of embodiments 50-56, wherein the subject did not receive prior osimertinib therapy.
58. The method of any one of embodiments 50-56, wherein the subject did not progress while on prior osimertinib therapy.
59. The method of any one of embodiments 1-58, wherein the c-Met IHC is performed according to the c-Met Teliso-V Staining Protocol.
60. The method of any one of embodiments 1-58, wherein ≥25% of tumor cells from tumor tissue of the c-Met expressing non-squamous NSCLC from the subject have ≥2+ membrane and/or cytoplasmic staining when assessed by c-Met IHC.
61. The method of any one of embodiments 1-58, wherein ≥25% of tumor cells from tumor tissue of the c-Met expressing non-squamous NSCLC from the subject have 3+ membrane and/or cytoplasmic staining when assessed by c-Met IHC.
62. The method of any one of embodiments 1-58, wherein ≥50% of tumor cells from tumor tissue of the c-Met expressing non-squamous NSCLC from the subject have 3+ membrane and/or cytoplasmic staining when assessed by c-Met IHC.
63. The method of any one of embodiments 1-62, wherein 1.6 mg/kg of Teliso-V is administered intravenously once every two weeks, and 80 mg of osimertinib is administered orally once per day.
64. The method of any one of embodiments 1-62, wherein 1.9 mg/kg of Teliso-V is administered intravenously once every two weeks, and 80 mg of osimertinib is administered orally once per day.
65. A method of treating a non-squamous non-small cell lung cancer (“NSCLC”) tumor that expresses c-Met, comprising administering to a human subject having said NSCLC tumor, wherein the human subject received previous osimertinib therapy and experienced progressive disease while on the osimertinib therapy
   1) osimertinib, and
   2) pharmaceutical composition comprising an anti-c-Met antibody drug conjugate (“ADC”), wherein the drug conjugate is monomethyl auristatin E (“MMAE”), and the ADC has the following structure:

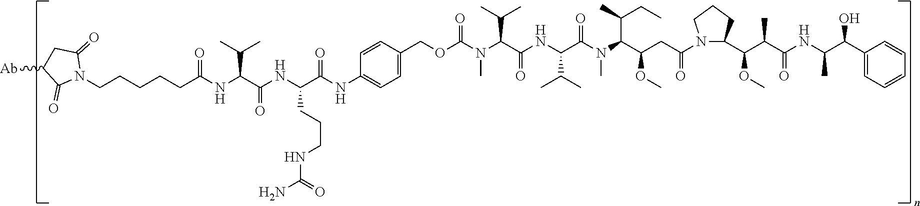

wherein Ab is an IgG antibody consisting of heavy chains each consisting of the amino acid sequence of SEQ ID NO:5 and light chains each consisting of the amino acid sequence of SEQ ID NO: 10, n has a value of 2 or 4, and attachment to the Ab is via a thioether linkage formed with a sulfhydryl group of a cysteine residue, and, wherein ≥25% of neoplastic cells from tumor tissue of the c-Met expressing non-squamous NSCLC from the subject have ≥1+ membrane and/or cytoplasmic staining when assessed by c-Met immunohistochemistry (IHC), wherein the NSCLC tumor carries a mutated EGFR gene;
wherein the mutated EGFR gene comprises an exon 19 deletion or an exon 21 L858R mutation
wherein the pharmaceutical composition comprising the anti-c-Met antibody drug conjugate has a drug antibody ratio (“DAR”) of about 2.4 to about 3.6
wherein 1.9 mg/kg of the anti-c-Met antibody drug conjugate is administered intravenously once every two weeks, and 80 mg of osimertinib is administered orally once per day.
66. The method of embodiment 65, wherein ≥25% of tumor cells from tumor tissue of the c-Met expressing non-squamous NSCLC from the subject have ≥2+ membrane and/or cytoplasmic staining when assessed by c-Met IHC.
67. The method of embodiment 65, wherein ≥25% of tumor cells from tumor tissue of the c-Met expressing non-squamous NSCLC from the subject have 3+ membrane and/or cytoplasmic staining when assessed by c-Met IHC.
68. The method of embodiment 65, wherein ≥50% of tumor cells from tumor tissue of the c-Met expressing non-squamous NSCLC from the subject have 3+ membrane and/or cytoplasmic staining when assessed by c-Met IHC.
69. The method of embodiment 65, wherein the tumor tissue is taken prior to administration of the first dose of the anti-c-Met antibody drug conjugate.
70. The method of embodiment 65, wherein the subject has received prior systemic therapy in the locally advanced or metastatic setting.
71. The method of embodiment 65, wherein the c-Met IHC is performed according to the c-Met Teliso-V Staining Protocol.

What is claimed:

1. A method of treating a non-squamous non-small cell lung cancer (“NSCLC”) tumor that expresses c-Met, comprising administering to a human subject having said NSCLC tumor, wherein the human subject received previous osimertinib therapy and experienced progressive disease while on the osimertinib therapy
1) osimertinib, and
2) pharmaceutical composition comprising an anti-c-Met antibody drug conjugate (“ADC”), wherein the drug conjugate is monomethyl auristatin E (“MMAE”), and the ADC has the following structure: 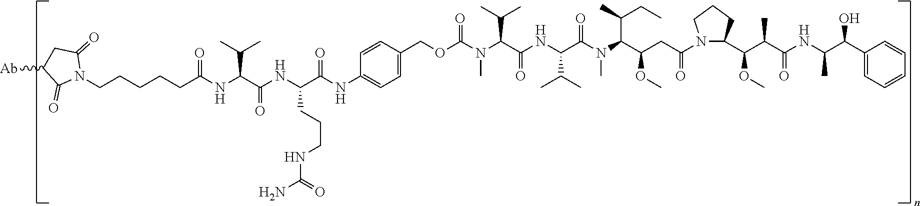
wherein Ab is an IgG antibody consisting of heavy chains each consisting of the amino acid sequence of SEQ ID NO:5 and light chains each consisting of the amino acid sequence of SEQ ID NO: 10, n has a value of 2 or 4, and attachment to the Ab is via a thioether linkage formed with a sulfhydryl group of a cysteine residue, and, wherein ≥25% of neoplastic cells from tumor tissue of the c-Met expressing non-squamous NSCLC from the subject have ≥1+ membrane and/or cytoplasmic staining when assessed by c-Met immunohistochemistry (IHC), wherein the NSCLC tumor carries a mutated EGFR gene.

2. The method of claim 1, wherein the mutated EGFR gene comprises an exon 19 deletion or an exon 21 L858R mutation.

3. The method of claim 2, wherein the mutated EGFR gene comprises a T790M mutation.

4. The method claim 1, wherein ≥25% of tumor cells from tumor tissue of the c-Met expressing non-squamous NSCLC from the subject have ≥2+ membrane and/or cytoplasmic staining when assessed by c-Met IHC.

5. The method claim 1, wherein ≥25% of tumor cells from tumor tissue of the c-Met expressing non-squamous NSCLC from the subject have 3+ membrane and/or cytoplasmic staining when assessed by c-Met IHC.

6. The method of claim 1, wherein ≥50% of tumor cells from tumor tissue of the c-Met expressing non-squamous NSCLC from the subject have 3+ membrane and/or cytoplasmic staining when assessed by c-Met IHC.

7. The method of claim 1, wherein administration of osimertinib and the anti-c-Met antibody drug conjugate achieves an objective response rate (ORR) in the subject that is greater than 25%, greater than 30%, greater than 35%, greater than 40%, greater than 45%, greater than 50%, greater than 55%, greater than 60%, greater than 65%, or greater than 70% or more.

8. The method of claim 1, wherein administration of osimertinib and the anti-c-Met antibody drug conjugate achieves a median duration of response (DoR) in the subject of at least 4 months, at least 6 months, at least 8 months, or at least 10 months.

9. The method of claim 1, wherein administration of osimertinib and the anti-c-Met antibody drug conjugate achieves progression free survival (PFS) in the subject of at least 5 months, at least 5.5 months, at least 6.5 months or at least 7.5 months.

10. The method of claim 1, wherein administration of osimertinib and the anti-c-Met antibody drug conjugate achieves an overall survival (OS) in the subject of at least 16 months, or an OS of at least 19 months.

11. The method of claim 1, wherein administration of osimertinib and the anti-c-Met antibody drug conjugate achieves a partial response (PR) in the subject.

12. The method of claim 1, wherein administration of osimertinib and the anti-c-Met antibody drug conjugate achieves a complete response (CR) in the subject.

13. The method of claim 1, wherein administration of osimertinib and the anti-c-Met antibody drug conjugate achieves an objective response rate (ORR) in the subject that is greater than 25%, preferably greater than 35%, and a median duration of response (DoR) in the subject of at least 4 months.

14. The method of claim 1, wherein the pharmaceutical composition comprising the anti-c-Met antibody drug conjugate has a drug antibody ratio (“DAR”) of about 2.4 to about 3.6.

15. The method of claim 1, wherein the pharmaceutical composition comprising the anti-c-Met antibody drug conjugate has a drug antibody ratio (“DAR”) of about 2.9 to about 3.1.

16. The method of claim 1, wherein 1.6 or 1.9 mg/kg of the anti-c-Met antibody drug conjugate is administered intravenously once every two weeks, and 80 mg of osimertinib is administered orally once per day.

17. The method of claim 1, wherein 1.9 mg/kg of the anti-c-Met antibody drug conjugate is administered intravenously once every two weeks to subjects weighing 100 kg or less, and 190 mg is administered intravenously to subjects weighing over 100 kg.

18. The method of claim 1, wherein the tumor tissue is taken prior to administration of the first dose of the anti-c-Met antibody drug conjugate.

19. The method of claim 1, wherein the subject has received prior systemic therapy in the locally advanced or metastatic setting.

20. The method of claim 1, wherein the c-Met IHC is performed according to the c-Met Teliso-V Staining Protocol.

21. A method of treating a non-squamous non-small cell lung cancer (“NSCLC”) tumor that overexpresses c-Met in a human subject, wherein the subject received previous osimertinib therapy and experienced progressive disease while on the osimertinib therapy, comprising determining whether the tumor exhibits c-Met overexpression, wherein c-Met overexpression is defined by ≥25% of the neoplastic cells from tumor tissue of the non-squamous NSCLC having 3+ membrane and/or cytoplasmic staining when assessed by IHC; and if the tumor tissue exhibits c-Met overexpression, administering to the subject having said NSCLC tumor 1) osimertinib and 2) a pharmaceutical composition comprising an anti-c-Met antibody drug conjugate (“ADC”), wherein the drug conjugate is monomethyl auristatin E (“MMAE”), and the ADC has the following structure: 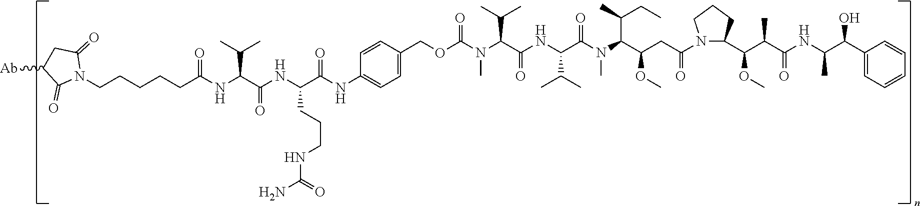
wherein Ab is an IgG antibody consisting of heavy chains each consisting of the amino acid sequence of SEQ ID NO:5 and light chains each consisting of the amino acid sequence of SEQ ID NO: 10, n has a value of 2 or 4, and attachment to the Ab is via a thioether linkage formed with a sulfhydryl group of a cysteine residue wherein the non-squamous NSCLC carries a mutated EGFR gene.

22. The method of claim 21, wherein the mutated EGFR gene comprises an exon 19 deletion or an exon 21 L858R mutation, and wherein administration of osimertinib and the anti-c-Met antibody drug conjugate achieves an objective response rate (ORR) in the subject that is greater than 25%, preferably greater than 35%, optionally wherein administration of osimertinib and the anti-c-Met antibody drug conjugate achieves a median duration of response (DoR) in the subject of at least 4 months.

23. The method of claim 21, wherein 80 mg of osimertinib is administered orally once per day, and 1.6 or 1.9 mg/kg of the anti-c-Met antibody drug conjugate is administered intravenously once every two weeks, and the pharmaceutical composition comprising the the anti-c-Met antibody drug conjugate has a drug antibody ratio (“DAR”) of about 2.4 to about 3.6.

24. A method of treating non-squamous non-small cell lung cancer (“NSCLC”) tumors that express c-Met in a plurality of human subjects, comprising the steps of:
(a) determining whether the tumor exhibits: i) c-Met overexpression or ii) lack of c-Met overexpression, wherein
i) c-Met overexpression is defined by ≥25% of the neoplastic cells from tumor tissue of the non-squamous NSCLC having 3+ membrane and/or cytoplasmic staining when assessed by IHC;
ii) lack of c-Met overexpression is defined by <25% of the neoplastic cells from tumor tissue of the non-squamous NSCLC having 3+ membrane and/or cytoplasmic staining when assessed by IHC;
(b) if the tumor tissue exhibits lack of c-Met overexpression, excluding the subject having the tumor that exhibits lack of c-Met overexpression from treatment;
(c) if the tumor tissue exhibits c-Met overexpression, selecting the subject for treatment and administering to the selected subject 1) osimertinib and 2) a pharmaceutical composition comprising an anti-c-Met antibody drug conjugate (“ADC”), wherein the drug conjugate is monomethyl auristatin E (“MMAE”), and the ADC has the following structure: 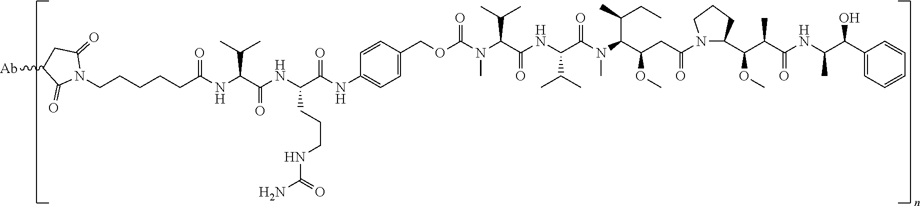
wherein Ab is an IgG antibody consisting of heavy chains each consisting of the amino acid sequence of SEQ ID NO:5 and light chains each consisting of the amino acid sequence of SEQ ID NO: 10, n has a value of 2 or 4, and attachment to the Ab is via a thioether linkage formed with a sulfhydryl group of a cysteine residue, wherein the non-squamous NSCLC tumor carries a mutated EGFR gene.

25. The method of claim 24, wherein the mutated EGFR gene comprises an exon 19 deletion or an exon 21 L858R mutation, and wherein administration of osimertinib and the anti-c-Met antibody drug conjugate achieves an objective response rate (ORR) in the selected subject that is greater than 25% and a median duration of response (DoR) in the subject of at least 4 months.

26. The method of claim 24, wherein 80 mg of osimertinib is administered orally once per day, and 1.6 or 1.9 mg/kg of the anti-c-Met antibody drug conjugate is administered intravenously once every two weeks, and the pharmaceutical composition comprising the anti-c-Met antibody drug conjugate has a drug antibody ratio (“DAR”) of about 2.4 to about 3.6.

27. A method of treating a non-squamous non-small cell lung cancer (“NSCLC”) tumor that expresses c-Met, comprising administering to a human subject having said NSCLC tumor, wherein the human subject received previous osimertinib therapy and experienced progressive disease while on the osimertinib therapy
1) osimertinib, and
2) pharmaceutical composition comprising an anti-c-Met antibody drug conjugate (“ADC”), wherein the drug conjugate is monomethyl auristatin E (“MMAE”), and the ADC has the following structure: 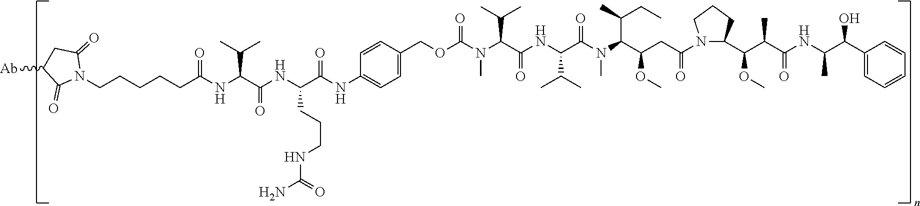
wherein Ab is an IgG antibody consisting of heavy chains each consisting of the amino acid sequence of SEQ ID NO:5 and light chains each consisting of the amino acid sequence of SEQ ID NO: 10, n has a value of 2 or 4, and attachment to the Ab is via a thioether linkage formed with a sulfhydryl group of a cysteine residue, and, wherein ≥25% of neoplastic cells from tumor tissue of the c-Met expressing non-squamous NSCLC from the subject have ≥1+ membrane and/or cytoplasmic staining when assessed by c-Met immunohistochemistry (IHC), wherein the NSCLC tumor carries a mutated EGFR gene;
wherein the mutated EGFR gene comprises an exon 19 deletion or an exon 21 L858R mutation
wherein the pharmaceutical composition comprising the anti-c-Met antibody drug conjugate has a drug antibody ratio (“DAR”) of about 2.4 to about 3.6
wherein 1.6 or 1.9 mg/kg of the anti-c-Met antibody drug conjugate is administered intravenously once every two weeks, and 80 mg of osimertinib is administered orally once per day.

28. The method of claim 27, wherein ≥25% of tumor cells from tumor tissue of the c-Met expressing non-squamous NSCLC from the subject have ≥2+ membrane and/or cytoplasmic staining when assessed by c-Met IHC.

29. The method of claim 27, wherein ≥25% of tumor cells from tumor tissue of the c-Met expressing non-squamous NSCLC from the subject have 3+ membrane and/or cytoplasmic staining when assessed by c-Met IHC.

30. The method of claim 27, wherein ≥50% of tumor cells from tumor tissue of the c-Met expressing non-squamous NSCLC from the subject have 3+ membrane and/or cytoplasmic staining when assessed by c-Met IHC.

31. The method of claim 27, wherein the tumor tissue is taken prior to administration of the first dose of the anti-c-Met antibody drug conjugate.

32. The method of claim 27, wherein the subject has received prior systemic therapy in the locally advanced or metastatic setting.

33. The method of claim 27, wherein the c-Met IHC is performed according to the c-Met Teliso-V Staining Protocol.


SEQUENCE LISTING (18 sequences)

SEQ ID NO: 1 - protein, 10 aa, synthetic construct
  REGION 1..10: note = Synthetic polypeptide
GYIFTAYTMH

SEQ ID NO: 2 - protein, 17 aa, synthetic construct
  REGION 1..17: note = Synthetic polypeptide
WIKPNNGLANYAQKFQG

SEQ ID NO: 3 - protein, 9 aa, synthetic construct
  REGION 1..9: note = Synthetic polypeptide
SEITTEFDY

SEQ ID NO: 4 - protein, 118 aa, synthetic construct
  REGION 1..118: note = Synthetic polypeptide
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 5 - protein, 445 aa, synthetic construct
  REGION 1..445: note = Synthetic polypeptide
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 15 aa, synthetic construct
  REGION 1..15: note = Synthetic polypeptide
KSSESVDSYANSFLH

SEQ ID NO: 7 - protein, 7 aa, synthetic construct
  REGION 1..7: note = Synthetic polypeptide
RASTRES

SEQ ID NO: 8 - protein, 9 aa, synthetic construct
  REGION 1..9: note = Synthetic polypeptide
QQSKEDPLT

SEQ ID NO: 9 - protein, 111 aa, synthetic construct
  REGION 1..111: note = Synthetic polypeptide
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 10 - protein, 218 aa, synthetic construct
  REGION 1..218: note = Synthetic polypeptide
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1395 bp, synthetic construct
  misc_feature 1..1395: note = Synthetic polynucleotide
  source 1..1395: mol_type = other DNA
atgggatggtcttggatctttctgctgtttctgtctggtactgctggtgtgctgagccaggtccagctggtgcaatccggcgcagaggtgaagaagccaggcgcttccgtgaaggtgagctgtaaggcctctggctacatcttcacagcatacaccatgcactgggtgaggcaagctcctgggcagggactggagtggatgggatggattaaacccaacaatgggctggccaactacgcccagaaattccagggtagggtcactatgacaagggataccagcatcagcaccgcatatatggagctgagcaggctgaggtctgacgacactgctgtctattattgcgccaggagcgaaattacaacagaattcgattactgggggcagggcaccctggtgaccgtgtcctctgccagcaccaagggcccaagcgtgttccccctggcccccagcagcaagagcaccagcggcggcacagccgccctgggctgcctggtgaaggactacttccccgagcccgtgaccgtgtcctggaacagcggagccctcacttctggagttcataccttcccagcagtattgcagagcagtggcctgtattcactgtcttccgtcgtaacagttccatcctccagcctcgggacacagacttacatttgtaacgtgaatcacaagcctagcaacaccaaggtcgacaagagagttgaaccaaagagttgtgattgccactgtcctccctgcccagctcctgagctgcttggcggtcccagtgtcttcttgtttccccctaaacccaaagacaccctgatgatctcaaggactcccgaggtgacatgcgtggtggtggatgtgtctcatgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggagtacaagtgtaaggtgtccaacaaggccctgccagccccaatcgaaaagaccatcagcaaggccaagggccagccaagagagccccaggtgtacaccctgccacccagcagggaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaagctgaccgtggacaagagcagatggcagcagggcaacgtgttcagctgctccgtgatgcacgaggccctgcacaaccactacacccagaagagcctgagcctgtccccaggctga

SEQ ID NO: 12 - DNA, 30 bp, synthetic construct
  misc_feature 1..30: note = Synthetic polynucleotide
  source 1..30: mol_type = other DNA
ggctacatcttcacagcatacaccatgcac

SEQ ID NO: 13 - DNA, 51 bp, synthetic construct
  misc_feature 1..51: note = Synthetic polynucleotide
  source 1..51: mol_type = other DNA
tggattaaacccaacaatgggctggccaactacgcccagaaattccagggt

SEQ ID NO: 14 - DNA, 27 bp, synthetic construct
  misc_feature 1..27: note = Synthetic polynucleotide
  source 1..27: mol_type = other DNA
agcgaaattacaacagaattcgattac

SEQ ID NO: 15 - DNA, 508 bp, synthetic construct
  misc_feature 1..508: note = Synthetic polynucleotide
  source 1..508: mol_type = other DNA
gctgatttacagggcttctaccagggagagcggcgtgcccgataggttcagcggatctggcagcggcaccgactttacactgaccatctccagcctgcaggccgaagatgtggcagtctattactgccagcagtccaaggaggaccccctgactttcgggggtggtactaaagtggagatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgctga

SEQ ID NO: 16 - DNA, 45 bp, synthetic construct
  misc_feature 1..45: note = Synthetic polynucleotide
  source 1..45: mol_type = other DNA
aaaagctccgaatctgtggactcttacgcaaacagctttctgcac

SEQ ID NO: 17 - DNA, 21 bp, synthetic construct
  misc_feature 1..21: note = Synthetic polynucleotide
  source 1..21: mol_type = other DNA
agggcttctaccagggagagc

SEQ ID NO: 18 - DNA, 27 bp, synthetic construct
  misc_feature 1..27: note = Synthetic polynucleotide
  source 1..27: mol_type = other DNA
cagcagtccaaggaggaccccctgact